(19) United States
(12) Patent Application Publication
(10) Pub. No.: US 2023/0404998 A1
(43) Pub. Date: Dec. 21, 2023

(54) Combination Therapies for Treating Cancer

(71) Applicants: ASCENTAGE PHARMA (SUZHOU) CO., LTD., SUZHOU (CN); ASCENTAGE PHARMA GROUP CORP LIMITED, CENTRAL (CN); GUANGZHOU HEALTHQUEST PHARMA CO., LTD., Guangzhou (CN)

(72) Inventors: Yifan ZHAI, Suzhou (CN); Dajun YANG, Suzhou (CN); Guangfeng WANG, Suzhou (CN); Miaozhen QIU, Suzhou (CN); Fan LUO, Suzhou (CN)

(21) Appl. No.: 18/298,800

(22) Filed: Apr. 11, 2023

Related U.S. Application Data
(63) Continuation-in-part of application No. PCT/CN2021/131503, filed on Nov. 18, 2021.

(30) Foreign Application Priority Data
Nov. 19, 2020 (WO) PCT/CN2020/130149
Nov. 24, 2020 (WO) PCT/CN2020/131184
Mar. 17, 2021 (WO) PCT/CN2021/081370

(51) Int. Cl. A61K 31/496; A61K 31/4439; A61K 39/395; A61P 35/02

(52) U.S. Cl. CPC A61K 31/496; A61K 31/4439; A61K 39/3955; A61P 35/02; A61K 2039/505

(57) ABSTRACT

The present invention relates to one or more combination treatments of cancer patients with a compound of formula (I),

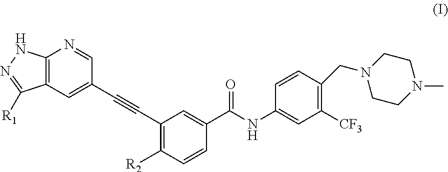

and an allosteric inhibitor or an immune checkpoint molecule, wherein R₁ and R₂ are as described herein.

### CROSS-REFERENCE TO RELATED APPLICATIONS

This application is a continuation-in-part of International Application No. PCT/CN2021/131503 filed Nov. 18, 2021, which claims priority to Chinese Patent Application No. PCT/CN2021/081370, filed Mar. 17, 2021, which also claims priority to Chinese Patent Application No. PCT/CN2020/131184, filed Nov. 24, 2020, and which claims priority to Chinese Patent Application No. PCT/CN2020/130149, filed Nov. 19, 2020, all applications filed with the Chinese State Intellectual Property Office. The entire contents of the aforementioned applications are incorporated herein by reference in their entireties.

### REFERENCE TO THE SEQUENCE LISTING

The instant application contains a Sequence Listing which has been submitted electronically in XML format and which is hereby incorporated by reference in its entirety. Said XML copy, created on Sep. 5, 2023 is named 266782-525993_Sequence-listing and is 6,344 bytes in size.

### FIELD OF THE INVENTION

The present invention relates to one or more combination treatments of cancer patients with a compound of formula (I), as described herein, and an allosteric inhibitor or an immune checkpoint molecule.

### BACKGROUND OF THE INVENTION

Chronic myeloid leukemia (CML) is a rare hematologic malignancy with an annual incidence rate of approximately 1.9 cases/100,000. BCR-ABL tyrosine kinase inhibitors (TKIs) have significantly improved clinical management of CML. However, despite clinical benefits offered by the first-generation BCR-ABL TKI imatinib (Gleevec®), and several second-generation TKIs, many patients develop drug resistance. Such acquired resistance to TKIs is a major challenge in the treatment of CML. BCR-ABL kinase mutations represent a key mechanism of acquired drug resistance; T315I, which is the most common drug-resistant mutation, occurs in about 25% of patients with drug-resistant CML. Patients with the T315I mutation are resistant to both first- and second-generation BCR-ABL inhibitors. Accordingly, there is a continuing need for new therapies and treatments that are more effective. The methods of the present invention present cancer patients with new options.

HQP-1351 is a novel, orally active, potent third-generation BCR-ABL inhibitor designed to effectively target BCR-ABL mutants, including T315I, and it is being developed for the treatment of patients with CML resistant to first- and second-generation TKIs.

### SUMMARY OF THE INVENTION

In one aspect, the present invention provides a method for treating cancer, the method comprises co-administering to a subject in need thereof:
a) a compound of formula (I) or a pharmaceutically acceptable salt thereof; and
b) an allosteric inhibitor;
wherein formula (I) has the following structure:

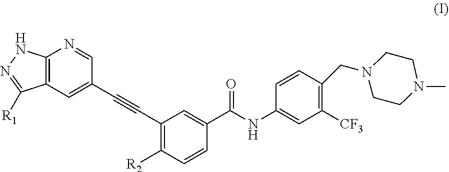

wherein
R1 is hydrogen, C1-4 alkyl, C3-6 cycloalkyl, C1-4 alkyloxy, or phenyl; and
R2 is hydrogen, C1-4 alkyl, C3-6 cycloalkyl, or halogen.

In another aspect, the present invention provides a method for treating cancer, the method comprises co-administering to a subject in need thereof
a) a compound of formula (I) or a pharmaceutically acceptable salt thereof; and
b) an immune checkpoint molecule.

In one embodiment, the compound of formula (I) is HQP-1351 having the following structure:

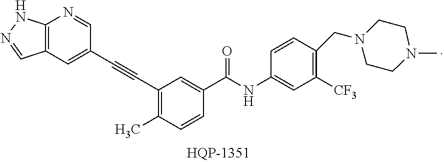 [SMILES: Cc1ccc(C(=O)Nc2ccc(CN3CCN(C)CC3)c(C(F)(F)F)c2)cc1C#Cc1cnc2[nH]ncc2c1]

In one embodiment, the allosteric inhibitor is asciminib.

In one embodiment, the immune checkpoint molecule is PD-1 or PD-L1.

### BRIEF DESCRIPTION OF FIGURES

FIG. 1A illustrates the results of ABL001 only or +HQP-1351 in BaF3(Bcr-Abl, T315I/F317L) cells in the WST assay.

FIG. 1B illustrates the results of ABL001 only or +Ponatinib in BaF3(Bcr-Abl, T315I/F317L) cells in the WST assay.

FIG. 2A illustrates the results of HQP-1351 only or +ABL001 in BaF3(Bcr-Abl, T315I/Y253) cells in the WST assay.

FIG. 2B illustrates the results in Ponatinib only or +ABL001 in BaF3(Bcr-Abl, T315I/Y253) cells in the WST assay.

FIG. 2C illustrates the results of ABL001 only or +HQP-1351, in BaF3(Bcr-Abl, T315I/Y253) cells in the WST assay.

FIG. 2D illustrates the results in ABL001 only or +Ponatinib in BaF3(Bcr-Abl, T315I/Y253) cells in the WST assay.

FIG. 3A illustrates the results of ABL001 only or +HQP-1351 in BaF3(Bcr-Abl,E255V/T315I) cells in the WST assay.

FIG. 3B illustrates the results of ABL001 only or +Ponatinib in BaF3(Bcr-Abl,E255V/T315I) cells in the WST assay.

FIG. 4A illustrates the results of ABL001 only or +HQP-1351 in BaF3(Bcr-Abl,E255K/T315I) cells in the WST assay.

FIG. 4B illustrates the results of ABL001 only or +Ponatinib in BaF3(Bcr-Abl,E255K/T315I) cells in the WST assay.

FIG. 5 illustrates WB-TZ-04-2020-HQP-1351, Ponatinib+ABL001 in BaF3 Bcr-Abl-T315I cell study results.

FIG. 6 illustrates WB-TZ-04-2020-HQP-1351, Ponatinib+ABL001 in BaF3 Bcr-Abl-E255V/T315I cell lines-20201110-1111 study results.

FIG. 7A illustrates HQP-1351, Ponatinib+ABL001 induced apoptosis in BaF3 Bcr-Abl-E255V/T315I cell lines-20201111-1113 by flow cytometry analysis.

FIG. 7B illustrates HQP-1351, Ponatinib+ABL001 induced apoptosis in BaF3 Bcr-Abl-E255V/T315I cell lines-20201111-1113 by Western blot.

FIG. 8A illustrates superior effect when HQP-1351 is combined with ABL001 in a T315I orthotopic tumor mouse model.

FIG. 8B illustrates superior effect when HQP-1351 is combined with ABL001 in a T315I orthotopic tumor mouse model.

FIG. 9A illustrates superior effect when HQP-1351 is combined with ABL001 in a Y253H/T315I orthotopic tumor mouse model.

FIG. 9B illustrates superior effect when HQP-1351 is combined with ABL001 in a Y253H/T315I orthotopic tumor mouse model.

FIG. 10A illustrates superior efficacy of HQP-1351 and ALB001 in a F317L/T315I orthotopic tumor mouse model.

FIG. 10B illustrates superior efficacy of HQP-1351 and ALB001 in a F317L/T315I orthotopic tumor mouse model.

FIG. 11A illustrates comparable efficacy of HQP-1351 and ALB001 in a E255V/T315I orthotopic tumor mouse model.

FIG. 11B illustrates comparable efficacy of HQP-1351 and ALB001 in a E255V/T315I orthotopic tumor mouse model.

FIG. 12A illustrates that HQP-1351 potentiates anti-PD-1 efficacy in vitro.

FIG. 12B illustrates that HQP-1351 potentiates anti-PD-1 efficacy in vitro.

FIG. 12C illustrates that HQP-1351 potentiates anti-PD-1 efficacy in vitro.

FIG. 13A illustrates that HQP-1351 potentiates anti-PD-L1 efficacy in vivo.

FIG. 13B illustrates that HQP-1351 potentiates anti-PD-L1 efficacy in vivo.

FIG. 13C illustrates that HQP-1351 potentiates anti-PD-L1 efficacy in vivo.

FIG. 13D illustrates that HQP-1351 potentiates anti-PD-L1 efficacy in vivo.

FIG. 13E illustrates that HQP-1351 potentiates anti-PD-L1 efficacy in vivo.

FIG. 13F illustrates that HQP-1351 potentiates anti-PD-L1 efficacy in vivo.

FIG. 14A illustrates that HQP-1351 suppress p-SRC and PD-L1 expression in a dose- and time-dependent manner.

FIG. 14B illustrates that HQP-1351 suppress p-SRC and PD-L1 expression in a dose- and time-dependent manner.

FIG. 14C illustrates that HQP-1351 suppress p-SRC and PD-L1 expression in a dose- and time-dependent manner.

FIG. 14D illustrates that HQP-1351 suppress p-SRC and PD-L1 expression in a dose- and time-dependent manner.

FIG. 14E illustrates that HQP-1351 suppress p-SRC and PD-L1 expression in a dose- and time-dependent manner.

FIG. 14F illustrates that HQP-1351 suppress p-SRC and PD-L1 expression in a dose- and time-dependent manner.

FIG. 15 shows cell viability curve of SUP-B15 cells treated with HQP-1351 for 72 hours.

FIG. 16 shows the anti-proliferation IC50 values of HQP-1351 in Philadelphia.

### DETAILED DESCRIPTION OF THE INVENTION

All published documents cited herein are hereby incorporated herein by reference in their entirety.

Unless defined otherwise, all technical and scientific terms used herein have the same meaning as commonly understood by one of ordinary skill in the art to which this disclosure belongs.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

The term “comprises” refers to “includes, but is not limited to.”

The terms “treatment,” “treat,” and “treating” refer to reversing, alleviating, delaying the onset of, or inhibiting the progress of a disease or disorder, or one or more symptoms thereof, including but not limited to therapeutic benefit. In some embodiments, treatment is administered after one or more symptoms have developed. In some embodiments, treatment may be administered in the absence of symptoms. For example, treatment may be administered to a subject prior to the onset of symptoms (e.g., in light of a history of symptoms and/or in light of genetic or other susceptibility factors). Treatment may also be continued after symptoms have resolved, for example to prevent or delay their recurrence.

The term “ABL001” refers to asciminib.

Therapeutic benefit includes eradication and/or amelioration of the underlying disorder being treated such as cancer; it also includes the eradication and/or amelioration of one or more of the symptoms associated with the underlying disorder such that an improvement is observed in the subject, notwithstanding that the subject may still be afflicted with the underlying disorder.

In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life.)

The term, “administering” or “administration” encompasses the delivery to a patient a compound or a pharmaceutically acceptable salt thereof, or a prodrug or other pharmaceutically acceptable derivative thereof, using any suitable formulation or route of administration, e.g., as described herein.

The term “co-administration” or “combination therapy” is understood as administration of two or more active agents using separate formulations or a single pharmaceutical formulation, or consecutive administration in any order such that, there is a time period while both (or all) active agents simultaneously exert their biological activities.

The term “therapeutically effective amount” or “effective amount” refers to an amount that is effective to elicit the desired biological or medical response, including the amount of a compound that, when administered to a subject for treating a disorder, is sufficient to effect such treatment of the disorder. The effective amount will vary depending on the disorder, and its severity, and the age, weight, etc. of the subject to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

The term, “patient” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys).

A “subject” to be treated by the method of the invention can mean either a human or non-human animal, preferably a mammal, more preferably a human. In certain embodiments, a subject has a detectable tumor prior to initiation of treatments using the methods of the invention. In certain embodiments, the subject has a detectable tumor at the time of initiation of the treatments using the methods of the invention.

The term “Preventing” or “prevention” refers to a reduction in risk of acquiring a disease or disorder. Prevention does not require that the disease or condition never occur, or recur, in the subject.

The term, “pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of Compound 1 include those derived from suitable inorganic and organic acids and bases.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Although pharmaceutically acceptable counter ions will be preferred for preparing pharmaceutical formulations, other anions are quite acceptable as synthetic intermediates. Thus it may be pharmaceutically undesirable anions, such as iodide, oxalate, trifluoromethanesulfonate and the like, when such salts are chemical intermediates.

The term “pharmaceutically acceptable carrier” is used herein to refer to a material that is compatible with a recipient subject, preferably a mammal, more preferably a human, and is suitable for delivering an active agent to the target site without terminating the activity of the agent. The toxicity or adverse effects, if any, associated with the carrier preferably are commensurate with a reasonable risk/benefit ratio for the intended use of the active agent.

The term “orally” refers to administering a composition that is intended to be ingested. Examples of oral forms include, but are not limited to, tablets, pills, capsules, powders, granules, solutions or suspensions, and drops. Such forms may be swallowed whole or may be in chewable form.

The term an “immune checkpoint” or “immune checkpoint molecule” is a molecule in the immune system that modulates a signal. An immune checkpoint molecule can be a co-stimulatory checkpoint molecule, i.e., turn up a signal, or an inhibitory checkpoint molecule, i.e., turn down a signal. A “co-stimulatory checkpoint molecule” as used herein is a molecule in the immune system that turns up a signal or is co-stimulatory. An “inhibitory checkpoint molecule”, as used herein is a molecule in the immune system that turns down a signal or is co-inhibitory.

The term a “modulator of an immune checkpoint molecule” is an agent capable of altering the activity of an immune checkpoint in a subject. In certain embodiments, a modulator of an immune checkpoint molecule alters the function of one or more immune checkpoint molecules including PD-1, PD-L1, PD-L2, CTLA-4, TIM-3, LAG3, CD160, 2B4, TGF β, VISTA, BTLA, TIGIT, LAIR1, OX40, CD2, CD27, ICAM-1, NKG2C, SLAMF7, NKp80, CD160, B7-H3, LFA-1,1COS, 4-1BB, GITR, CD30, CD40, BAFFR, HVEM, CD7, LIGHT, and CD83. The modulator of the immune checkpoint may be an activator (e.g., an agonist) or an inhibitor (e.g., an antagonist) of the immune checkpoint. In some embodiments, the modulator of the immune checkpoint molecule is an immune checkpoint binding protein (e.g., an antibody, antibody Fab fragment, divalent antibody, antibody drug conjugate, scFv, fusion protein, bivalent antibody, or tetravalent antibody). In some embodiments, the modulator of the immune checkpoint molecule is a monoclonal antibody or an antigen binding fragment thereof. In other embodiments, the modulator of the immune checkpoint molecule is a small molecule. In a particular embodiment, the modulator of the immune checkpoint molecule is an anti-PD1 antibody. In a particular embodiment, the modulator of the immune checkpoint molecule is an anti-PD-L1 antibody. In a particular embodiment, the modulator of the immune checkpoint molecule is an anti-CTLA-4 antibody.

As used herein, the term “co-administering” or “co-administration” refers to administration of a compound of formula (I) or HQP-1351 prior to, concurrently or substantially concurrently with, subsequently to, or intermittently with the administration of an allosteric inhibitor or an immune checkpoint modulator.

The term “complete regression” refers to tumor is not detectable after treatment.

The term “partial regression” refers to tumor volumes become smaller (e.g., 10%, 20%, 30%, 40%, 50%, 60%, 70%, 80%, 90% less) compared to before treatment.

In all occurrences in this application where there are a series of recited numerical values, it is to be understood that any of the recited numerical values may be the upper limit or lower limit of a numerical range. It is to be further understood that the invention encompasses all such numerical ranges, i.e., a range having a combination of an upper numerical limit and a lower numerical limit, wherein the numerical value for each of the upper limit and the lower limit can be any numerical value recited herein. Ranges provided herein are understood to include all values within the range. For example, 1-10 is understood to include all of the values 1, 2, 3, 4, 5, 6, 7, 8, 9, and 10, and fractional values as appropriate. Ranges expressed as “up to” a certain value, e.g., up to 5, is understood as all values, including the upper limit of the range, e.g., 0, 1, 2, 3, 4, and 5, and fractional values as appropriate. Up to or within a week is understood to include, 0.5, 1, 2, 3, 4, 5, 6, or 7 days. Similarly, ranges delimited by “at least” are understood to include the lower value provided and all higher numbers.

The term “alkyl” means a branched-chain or straight chain alkyl group with the certain number of carbon atoms. For example, the definition of “C₁-C₅” in “C₁-C₅ alkyl” means straight-chain or branched-chain alkyl group with 1, 2, 3, 4 or 5 carbon atoms. For example, “C₁-C₅ alkyl” includes methyl, ethyl, n-propyl, isopropyl, n-butyl, tert-butyl, isobutyl, pentyl, etc.

The term “cycloalkyl” refers to a specific single saturated ring alkyl with the certain number of carbon atoms. For examples, “cycloalkyl” includes cyclopropyl-, methyl-cyclopropyl-, 2,2-dimethyl-cyclobutyl, 2-ethyl-cyclopentyl-, cyclohexyl etc.

The term “alkoxy” refers to methoxy, ethoxy, proposy, isopropoxy butoxy, isobutoxy, sec-butoxy, or tert-butoxy.

The term “halo” or “halogen” means chlorine, fluorine, bromine and iodine.

Various (enumerated) embodiments of the present invention are described herein. It will be recognized that features specified in each embodiment may be combined with other specified features to provide further embodiments of the present invention.

Embodiment 1 A method of treating cancer, comprising co-administering to a subject in need thereof:
a) a compound of formula (I) or a pharmaceutically acceptable salt thereof; and
b) an allosteric inhibitor;
wherein formula (I) has the following structure:

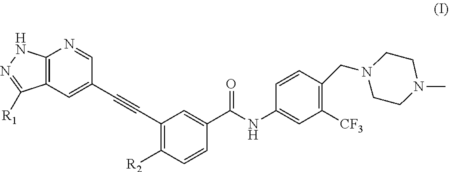

wherein
R₁ is hydrogen, C1-4 alkyl, C3-6 cycloalkyl, C1-4 alkyloxy, or phenyl; and
R₂ is hydrogen, C1-4 alkyl, C3-6 cycloalkyl, or halogen.

In one embodiment of the compound of formula (I), R₁ is hydrogen or C₁₋₄ alkyl.

In another embodiment of the compound of formula (I), R₁ is hydrogen.

In another embodiment of the compound of formula (I), R₂ is hydrogen or C₁₋₄ alkyl.

In another embodiment of the compound of formula (I), R₂ is C₁₋₄ alkyl.

In another embodiment of the compound of formula (I), R₂ is methyl or ethyl. In another embodiment, R₂ is methyl.

In another embodiment of the compound of formula (I), R₁ is hydrogen and R₂ is C₁₋₄ alkyl.

The compounds of formula (I) are novel, selective potent inhibitors against a broad spectrum of BCR-ABL mutations, including T315I, E255K/V, G250E, H396P, M351T, Q252H, Y253F/H, or BCR-ABL^{WT}.

The compounds of formula (I) or a pharmaceutically acceptable salt thereof are also potent inhibitors against other kinases including KIT, BRAF, DDR1, PDGFR, FGFR, FLT3, RET, SRC, TIE1, and TIE2.

Embodiment 2 The method of embodiment 1, wherein the compound of formula (I) is HQP-1351 or a pharmaceutically acceptable salt thereof, wherein HQP-1351 has the following structure:

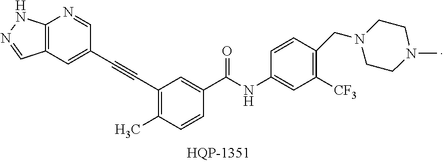 [SMILES: Cc1ccc(C(=O)Nc2ccc(CN3CCN(C)CC3)c(C(F)(F)F)c2)cc1C#Cc1cnc2[nH]ncc2c1]

HQP-1351 is a novel, orally active, potent third-generation BCR-ABL inhibitor designed to effectively target BCR-ABL mutants, including T315I, and it is being developed for the treatment of patients with CML resistant to first- and second-generation tyrosine kinase inhibitors (TKIs).

The chemical name for HQP-1351 is 3-(2-(1H-pyrazolo[3,4-b]pyridin-5-yl)ethynyl)-4-methyl-N-(4-((4-methylpiperazin-1-yl)methyl)-3-(trifluoromethyl)phenyl)-benzamide.

The compounds of formula (I) or HQP-1351 or a pharmaceutically acceptable salt thereof can be prepared according to the production methods described in U.S. Pat. No. 8,846,671 B2, issued Sep. 30, 2014, which is incorporated herein by reference in its entirety and for all purposes or a method analogous thereto.

Embodiment 3 The method of embodiment 1 or 2, wherein the allosteric inhibitor is asciminib.

The present invention discovers that HQP-1351 also known as olverembatinib, enhanced the effect of allosteric inhibitor on the resistance conferred by the compound mutations of BCR-ABL. Treatment with tyrosine kinase inhibitors (TKIs) directed against the ATP-binding site of BCR-ABL promotes recovery of Ph+ leukemia. However, emergence of check point mutation T315I and compound mutants confer resistance to these TKIs. HQP-1351 is a new generation TKI targeting BCR-ABL. It is an ATP-site inhibitor and currently in development for r/r CML.

Asciminib is an allosteric inhibitor targeting the myristoyl-binding pocket of BCR-ABL kinase and downstream signaling. Studies show that combining asciminib with ponatinib can overcome only a subset of the resistance caused by BCR-ABL compound mutants. A novel combination of HQP-1351 with asciminib, targeting both ATP pocket and allosteric region of BCR-ABL protein, can promote the inhibitory effect on the kinase harboring compound mutations.

Asciminib may be prepared according to the methods described in Nature (London, United Kingdom), 543(7647), 733-737: 2017, or according to the methods described in the PCT publication WO2013/171639.

A series of cells with BCR-ABL single or compound mutations were constructed based on BaF3 cells. Effect of HQP-1351 as a single agent or in combination with asciminib were analyzed using antiproliferation, Western blot and FACS assays in vitro. In vivo efficacy was evaluated using the syngeneic mouse model derived from BaF3 cells with T315I and/or compound mutations.

The cell based antiproliferation studies demonstrated superior activity of HQP-1351 toward BCR-ABL single or compound mutations with IC₅₀ values ranging between 6-300 nM. In particular, HQP-1351 was more effective than ponatinib, asciminib and other TKIs against those compound mutations. Moreover, the combination of HQP-1351 with asciminib was highly effective against BCR-ABL compound mutations, especially those containing T315I. In vivo studies further revealed that co-administration of HQP-1351 with asciminib resulted in significant prolongation of survival compared to single agents. Importantly, the antitumor effect was more potent than that of ponatinib plus asciminib in models harboring compound mutations. In terms of mechanism, the combined treatment synergistically downregulated phosphorylation of BCR-ABL and the downstream proteins CRKL and STAT5, and augmented cleavage of Caspase-3 and PARP-1, thus triggered apoptosis and subsequently enhanced the antitumor effects.

Results in Example 1 of the present invention shows that the combination of ATP binding site inhibitor HQP-1351 and allosteric inhibitor may have the best antitumor effect on tumor cells harboring single or compound mutations in BCR-ABL. This novel strategy may help to overcome the secondary compound mutations post the treatment with single TKIs.

Embodiment 4 The method of embodiments 1-3, wherein the cancer is hematological malignancy.

Embodiment 5 The method of embodiment 4, wherein the hematological malignancy is leukemia.

Embodiment 6 The method of embodiment 4, wherein the hematological malignancy is chronic myelogenous leukemia.

Embodiment 7 The method of embodiment 1-6, wherein the method is in the treatment of the patient with chronic myeloid leukemia resistant to current tyrosine kinase inhibitor therapies.

Embodiment 8 The method of embodiment 7, wherein the patient with chronic myeloid leukemia resistant to the current tyrosine kinase inhibitor therapies is caused by BCR-ABL mutations.

Embodiment 9 The method of embodiment 8, where the BCR-ABL mutation is T3151, E255K/V, G250E, H396P, M351T, Q252H, Y253F/H, or BCR-ABL^{WT} mutations.

Embodiment 10 The method of embodiment 8, where the BCR-ABL mutation is T3151 mutation.

Embodiment 11 The method embodiments 1-3, wherein the cancer is breast cancer, cervical cancer, ovarian cancer, endometrial cancer, prostate cancer, colon cancer, bladder cancer, bone metastasis, colorectal cancer, esophagus cancer, head and neck cancer, lung cancer, lung carcinoid tumor, or stomach carcinoma.

Embodiment 12 The method of embodiment 11, where the lung cancer is non-small cell lung cancer (NSCLC).

Embodiment 13 The method of embodiment 11, where the lung cancer is small cell lung cancer (SCLC).

Embodiment 14 A method of inhibiting BCR-ABL mutants, the method comprises contacting BCR-ABL mutants with a) a compound of formula (I) or a pharmaceutically acceptable salt thereof; and b) an allosteric inhibitor;
wherein the compound of formula (I) has the following structure:

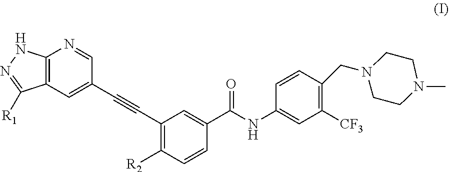

wherein
R₁ is hydrogen, C₁₋₄ alkyl, C₃₋₆ cycloalkyl, C₁₋₄ alkyloxy, or phenyl; and
R₂ is hydrogen, C₁₋₄ alkyl, C₃₋₆ cycloalkyl, of halogen.

Embodiment 15 The method of embodiment 14, wherein the compound of formula (I) is HQP-1351 or a pharmaceutically acceptable salt thereof, wherein HQP-1351 has the following structure:

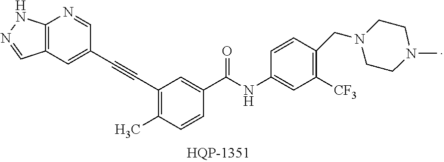 [SMILES: Cc1ccc(C(=O)Nc2ccc(CN3CCN(C)CC3)c(C(F)(F)F)c2)cc1C#Cc1cnc2[nH]ncc2c1]

Embodiment 16 The method of embodiments 14 or 15, wherein the allosteric inhibitor is asciminib.

Embodiment 17 The method of embodiments 14-16, wherein the inhibition is in vitro or in vivo.

Embodiment 18 The method of embodiments 14-19, wherein the inhibition is in a patient with chronic myeloid leukemia resistant to current tyrosine kinase inhibitor therapies.

Embodiment 19 The method of embodiment 18, wherein the patient with chronic myeloid leukemia resistant to the current tyrosine kinase inhibitor therapies is caused by BCR-ABL mutations.

Embodiment 20 The method of embodiment 19, wherein the BCR-ABL mutation is T3151, E255K/V, G250E, H396P, M351T, Q252H, Y253F/H, or BCR-ABL^{WT} mutations.

Embodiment 21 The method of embodiment 19, wherein the BCR-ABL mutation is T3151.

The present invention further discovers that HQP-1351 enhanced T-cell-mediated anti-tumor immune responses in NSCLC. The discovery of the present invention provides evidence that support the combination of HQP-1351 and anti-PD-1/PD-L1 as a potential combination therapeutic approach to increase the efficacy of immunotherapy in NSCLC.

Although immune checkpoint inhibitors (ICIs) included PD-1/PD-L1 antibody have demonstrated favorable therapeutic responses in some cancer treatments, a significant portion of patients with cancer remain non-responsive. Substantial limitations in the rate of non-response highlight the need for suitable combination therapies. SRC family kinases is overexpressed or activated in multiple human malignancies, as a candidate target in regulating antitumor immunity. HQP-1351, an orally bioavailable multi-kinase inhibitor, has exhibited clinical efficacy in chronic myeloid leukemia. The present invention demonstrates that HQP-1351 as a SRC inhibitor is capable of increasing the antitumor immunity in non-small cell lung cancer (NSCLC) as shown in Example below.

PD-1. Programmed cell death protein 1 (PD-1, also known as CD279 and PDCD1) is an inhibitory receptor that negatively regulates the immune system. In contrast to CTLA-4 which mainly affects naïve T cells, PD-1 is more broadly expressed on immune cells and regulates mature T cell activity in peripheral tissues and in the tumor microenvironment. PD-1 inhibits T cell responses by interfering with T cell receptor signaling. PD-1 has two ligands, PD-L1 and PD-L2. Multiple immune checkpoint modulators specific for PD-1 have been developed and may be used as disclosed herein. In some embodiments, the immune checkpoint modulator is an agent that modulates the activity and/or expression of PD-1. In some embodiments, the immune checkpoint modulator is an agent that binds to PD-1 (e.g., an anti-PD-1 antibody). In some embodiments, the checkpoint modulator is an PD-1 agonist. In some embodiments, the checkpoint modulator is an PD-1 antagonist. In some embodiments, the immune checkpoint modulator is a PD-1-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), JS-001 (Shanghai Junshi Bioscience Co., Ltd.), SHR-1210 (Incyte/Jiangsu Hengrui Medicine Co., Ltd.), MEDI0680 (also known as AMP-514; Amplimmune Inc./Medimmune), PDR001 (Novartis), BGB-A317 (BeiGene Ltd.), TSR-042 (also known as ANB011; AnaptysBio/Tesaro, Inc.), REGN2810 (also known as cemiplimab, Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-L1/PD-L2. PD ligand 1 (PD-L1, also known as B7-H1) and PD ligand 2 (PD-L2, also known as PDCD1LG2, CD273, and B7-DC) bind to the PD-1 receptor. Both ligands belong to the same B7 family as the B7-1 and B7-2 proteins that interact with CD28 and CTLA-4. PD-L1 can be expressed on many cell types including, for example, epithelial cells, endothelial cells, and immune cells. Ligation of PDL-1 decreases IFN, TNF, and IL-2 production and stimulates production of IL10, an anti-inflammatory cytokine associated with decreased T cell reactivity and proliferation as well as antigen-specific T cell anergy. PDL-2 is predominantly expressed on antigen presenting cells (APCs). PDL2 ligation also results in T cell suppression, but where PDL-i-PD-1 interactions inhibits proliferation via cell cycle arrest in the G1/G2 phase, PDL2-PD-1 engagement has been shown to inhibit TCR-mediated signaling by blocking B7:CD28 signals at low antigen concentrations and reducing cytokine production at high antigen concentrations. Multiple immune checkpoint modulators specific for PD-L1 and PD-L2 have been developed and may be used as disclosed herein.

In some embodiments, the immune checkpoint modulator is an agent that modulates the activity and/or expression of PD-L1. In some embodiments, the immune checkpoint modulator is an agent that binds to PD-L1 (e.g., an anti-PD-L1 antibody). In some embodiments, the checkpoint modulator is an PD-L1 agonist. In some embodiments, the checkpoint modulator is an PD-L1 antagonist. In some embodiments, the immune checkpoint modulator is a PD-L1-binding protein (e.g., an antibody or a Fc-fusion protein) selected from the group consisting of durvalumab (also known as MEDI-4736; AstraZeneca/Celgene Corp./Medimmune), atezolizumab (Tecentriq; also known as MPDL3280A and RG7446; Genetech Inc.), avelumab (also known as MSB0010718C; Merck Serono/AstraZeneca); MDX-1105 (BMS-936559, Medarex/Bristol-Meyers Squibb), AMP-224 (Amplimmune, GlaxoSmithKline), LY3300054 (Eli Lilly and Co.), JS003 (Shanghai Junshi Bioscience Co., Ltd.), SHR-1316 (Jiangsu Hengrui Medicine Co., Ltd.), KN035 (Alphamab and 3D Medicines), or CK-301 (Checkpoint Therapeutics). Additional PD-L1-binding proteins are known in the art and are disclosed, e.g., in U.S. Patent Application Publication Nos. 2016/0084839, 2015/0355184, 2016/0175397, and PCT Publication Nos. WO 2014/100079, WO 2016/030350, WO2013181634, each of which is incorporated by reference herein.

In some embodiments, the immune checkpoint modulator is an agent that modulates the activity and/or expression of PD-L2. In some embodiments, the immune checkpoint modulator is an agent that binds to PD-L2 (e.g., an anti-PD-L2 antibody). In some embodiments, the checkpoint modulator is an PD-L2 agonist. In some embodiments, the checkpoint modulator is an PD-L2 antagonist. PD-L2-binding proteins (e.g., antibodies) are known in the art and are disclosed, e.g., in U.S. Pat. Nos. 9,255,147, 8,188,238; U.S. Patent Application Publication Nos. 2016/0122431, 2013/0243752, 2010/0278816, 2016/0137731, 2015/0197571, 2013/0291136, 2011/0271358; and PCT Publication Nos. WO 2014/022758, and WO 2010/036959, each of which is incorporated by reference herein.

In certain embodiments, the administered dosage of the immune checkpoint modulator is 5%, 10%, 20%, 30%, 40%, 50%, 60%, 70%, 80%, or 90% lower than the standard dosage of the immune checkpoint modulator for a particular cancer. In certain embodiments, the dosage administered of the immune checkpoint modulator is 95%, 90%, 85%, 80%, 75%, 70%, 65%, 60%, 55%, 50%, 45%, 40%, 35%, 30%, 25%, 20%, 15%, 10% or 5% of the standard dosage of the immune checkpoint modulator for a particular oncological disorder. In one embodiment, where a combination of immune checkpoint modulators are administered, at least one of the immune checkpoint modulators is administered at a dose that is lower than the standard dosage of the immune checkpoint modulator for a particular oncological disorder. In one embodiment, where a combination of immune checkpoint modulators are administered, at least two of the immune checkpoint modulators are administered at a dose that is lower than the standard dosage of the immune checkpoint modulators for a particular oncological disorder. In one embodiment, where a combination of immune checkpoint modulators are administered, at least three of the immune checkpoint modulators are administered at a dose that is lower than the standard dosage of the immune checkpoint modulators for a particular oncological disorder. In one embodiment, where a combination of immune checkpoint modulators are administered, all of the immune checkpoint modulators are administered at a dose that is lower than the standard dosage of the immune checkpoint modulators for a particular oncological disorder. In some embodiments, the immune checkpoint modulator is administered at a dose that is lower than the standard dosage of the immune checkpoint modulator, and the compound of formula (I) or HQP-1351 is administered at a dose that is lower than the standard dosage.

Embodiment 22 A method of treating cancer, comprising co-administering to a subject in need thereof:
a) a compound of formula (I) or a pharmaceutically acceptable salt thereof; and
b) an immune checkpoint molecule, wherein formula (I) has the following structure:

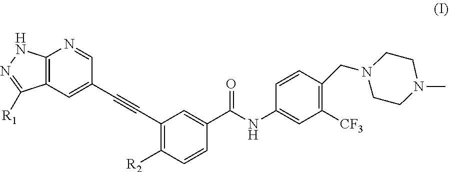

wherein
R₁ is hydrogen, C₁₋₄ alkyl, C₃₋₆ cycloalkyl, C₁₋₄ alkyloxy, or phenyl; and
R₂ is hydrogen, C₁₋₄ alkyl, C₃₋₆ cycloalkyl, of halogen.

Embodiment 23 The method of embodiment 22, wherein the compound of formula (I) is HQP-1351 or a pharmaceutically acceptable salt thereof.

Embodiment 24 The method of embodiments 21-22, wherein the immune checkpoint molecule is PD-1 or PD-L1.

Embodiment 25 The method of embodiments 21-22, wherein the immune checkpoint molecule is PD-1, PD-L1, PD-L2, CTLA-4, TIM-3, LAG3, CD160, 2B4, TGF 3, VISTA, BTLA, TIGIT or LAIR1.

Embodiment 26 The method of embodiments 21-22, wherein the immune checkpoint molecule is pembrolizumab, ipilimumab, nivolumab, atezolizumab, avelumab, durvalumab, cemiplimab, lirilumab, tremelimumab, or pidilizumab.

Embodiment 27 The method of embodiments 21-22, wherein the immune checkpoint molecule is AMP-224, AMP-514, BGB-A317, cemiplimab, JS001, PDR-001, PF-06801591, IBI-308, pidilizumab, SHR-1210, or TSR-042.

Embodiment 28 The method of embodiments 22-27, wherein the cancer is breast cancer, cervical cancer, ovarian cancer, endometrial cancer, prostate cancer, colon cancer, bladder cancer, bone metastasis, colorectal cancer, esophagus cancer, head and neck cancer, lung cancer, lung carcinoid tumor, or stomach carcinoma.

Embodiment 29 The method of embodiment 28, wherein the lung cancer is non-small cell lung cancer.

Embodiment 30 The method of embodiment 29, wherein the lung cancer is small cell lung cancer.

Embodiment 31 The method of embodiments 1-13 and 22-30, wherein the compound of formula (I), or pharmaceutically acceptable salt thereof is administered orally to the patients in need thereof.

Embodiment 32 The method of any one of claims 1-30, wherein the compound of formula (I), or pharmaceutically acceptable salt thereof is administered once every other day (QOD) during the 28-day treatment cycle.

Embodiment 33 The method of embodiment 32, wherein the compound of formula (I) is HQP-1351.

Embodiment 34 The method of embodiment 32, wherein HQP-1351 is administered once every other day in an amount of about 1 mg, about 2 mg, about 4 mg, or about 8 mg.

Embodiment 35 The method of embodiment 32, wherein HQP-1351 is administered once every other day in an amount of about 12 mg or about 20 mg.

Embodiment 36 The method of embodiment 32, wherein HQP-1351 is administered once every other day in an amount of about 30 mg, about 40 mg, or about 45 mg.

Embodiment 37 The method of embodiment 32, wherein HQP-1351 is administered once every other day in an amount of about 50 mg or about 60 mg.

Embodiment 38 A method of treating renal cancer comprising co-administering to a subject in need thereof:
a) a compound of formula (I) or a pharmaceutically acceptable salt thereof; and
b) an anti-PD-1 antibody, wherein formula (I) has the following structure:
wherein R₁ is hydrogen, C₁₋₄ alkyl, C₃₋₆ cycloalkyl, C₁₋₄ alkyloxy, or phenyl; and R₂ is hydrogen, C₁₋₄ alkyl, C₃₋₆ cycloalkyl, of halogen.

Embodiment 39 The method of embodiment 38, wherein the compound of formula (I) is HQP-1351 or a pharmaceutically acceptable salt thereof.

Embodiment 40 The method of embodiment 38 or 39, wherein the anti-PD-1 antibody is InVivoMab anti-mouse PD-1(CD279).

Embodiment 41 The method of embodiment 40, wherein the renal cancer is renal cell carcinoma.

Embodiment 42 The method of embodiment 41, where in the renal cell carcinoma is clear cell renal cell carcinoma (ccRCC).

Embodiment 43 The method of embodiment 41, where in the renal cell carcinoma is papillary renal cell carcinoma (PRCC).

Embodiment 44 The method of embodiment 41, where in the renal cell carcinoma is clear cell papillary renal cell carcinoma.

Embodiment 45 The method of embodiment 41, where in the renal cell carcinoma is chromophobe renal cell carcinoma.

In some embodiments, the combination therapy for treating cancer comprises at least one 21-day treatment cycle, wherein the compound of formula (I), such as HQP-1351, or pharmaceutically acceptable salt thereof, is administrated in an therapeutically effective amount.

In some embodiment, the therapeutically effective amount is from 0.5 mg to 100 mg, preferably from 1 mg to 80 mg, more preferably from 1 mg to 60 mg, most preferably about 1 mg, about 2 mg, about 4 mg, or about 8 mg.

In some embodiment, the therapeutically effective amount of HQP-1351 is about 12 mg or about 20 mg.

In some embodiment, the therapeutically effective amount of HQP-1351 is about 30 mg, about 40 mg, or about 45 mg.

In some embodiment, the therapeutically effective amount of HQP-1351 is about 50 mg or about 60 mg.

In some embodiment, the therapeutically effective amount of HQP-1351 is administered orally every other day in a patient in need thereof for the first two consecutive weeks of a 21-day treatment cycle and is not administered during the third week of the treatment cycle.

In some embodiments, the compound of formula (I), such as HQP-1351, or pharmaceutically acceptable salt thereof, is administered orally in the patient on day 1, 3, 5, 7, 9, 11, and 13 of the 21-day treatment cycle.

In some embodiments, the compound of formula (I), such as HQP-1351, or pharmaceutically acceptable salt thereof, is not administered on day 14-21 of the 21-day treatment cycle.

In some embodiments, the compound of formula (I), such as HQP-1351, or pharmaceutically acceptable salt thereof, is administered orally in the patient in an amount from about 50 mg to about 200 mg on day 1, 3, 5, 7, 9, 11, and 13 of the 21-day treatment cycle.

In some embodiments, the compound of formula (I), such as HQP-1351, or pharmaceutically acceptable salt thereof, is administered orally in the patient in an amount of about 50 mg on day 1, 3, 5, 7, 9, 11, and 13 of the 21-day treatment cycle.

In some embodiments, the compound of formula (I), such as HQP-1351, or pharmaceutically acceptable salt thereof, is administered orally in the patient in an amount of about 100 mg on day 1, 3, 5, 7, 9, 11, and 13 of the 21-day treatment cycle.

In some embodiments, the compound of formula (I), such as HQP-1351, or pharmaceutically acceptable salt thereof, is administered orally in the patient in an amount of about 150 mg on day 1, 3, 5, 7, 9, 11, and 13 of the 21-day treatment cycle.

In some embodiments, the compound of formula (I), such as HQP-1351, or pharmaceutically acceptable salt thereof, is administered orally in the patient in an amount of about 200 mg on day 1, 3, 5, 7, 9, 11, and 13 of the 21-day treatment cycle.

In some embodiments, as asciminib is administered orally.

In some embodiments, PD-1 or PD-L1 is administered via intravenous infusion in an amount of 200 mg on day 1 of the 21-day treatment cycle.

In some embodiments, the combination therapy comprises at least 1, 2, 3, 4, 5, 6, 7, 8, 9, 10, 11, or 12 of the 21-day treatment cycle. In some embodiments, the combination therapy continues until disease progression or unacceptable toxicity.

In certain embodiments, at least 1, 2, 3, 4, or 5 cycles of the combination therapy are administered to the subject. The subject is assessed for response criteria at the end of each cycle. The subject is also monitored throughout each cycle for adverse events (e.g., clotting, anemia, liver and kidney function, etc.) to ensure that the treatment regimen is being sufficiently tolerated.

The following examples are provided for purposes of illustration, not limitation.

### EXAMPLES

### Example 1 Anti-Proliferation Assay of HQP-1351 to Enhance the Effect of Allosteric Inhibitor on the Resistance Conferred by the Compound Mutations of BCR-ABL

### Methods

The anti-proliferative effect was determined by a water-soluble tetrazolium (WST)-based assay using Cell Counting Kit-8 (CCK-8, #D3100L4057, Shanghai life ilab bio technology co., LTD, China). Cells were seeded in 96-well plates and treated with different concentrations of test articles. The combination effect was tested using 9 different concentrations of asciminib with 3 fixed doses of HQP-1351. Each treatment was tested in triplicates. The plate was then cultured at 37° C. with 5% CO₂ for 72 hours. At the end of treatments, 20 μl/well of CCK-8 reagent was directly added to each wells. The plate was then incubated at 37° C. with 5% CO₂ for 2-4 h. The OD value was detected at 450 nm on a microplate reader (SpectraMax I3X, Molecular Devices, US). IC₅₀ values were calculated with Graphpad Prism 7.0 software using nonlinear regression (curve fitting) type data analysis. For combination effect assay, the cells viability was normalized using the mean OD value of triplicated wells for the single agent control. Synergy was indicated between the two compounds when the calculated IC₅₀ of the combinative curves are less than the IC₅₀ of single agent (the curves of combination shift left) and the combination index (CI) is less than 1.0 (Calcusyn Version 2.0, Zhou).

### Flow Cytometry Analysis

Apoptotic cells were determined by using an Annexin V-PI (propidium iodide) staining kit (#C1062L, Beyotime Biotechnology, China). Briefly, cells were harvested after treatments with test articles for indicated time, and washed with phosphate buffered saline (PBS). Cells were then stained with Annexin-V and PI, and analyzed with a flow cytometer (CytoFLEX, Beckman, US). Apoptotic profiles were obtained through analyzing 10,000 cells per treatment.

### Western Blotting Assay

After treatments with test articles for indicated time, cultured cells were harvested and washed with ice-cold PBS. Cell pellets were lysed in RIPA buffer containing 1% PMSF, and 1% protease inhibitors. Protein concentrations were determined using the BCA Protein Assay Kit (#P0011, Beyotime Biotechnology, China). Cell lysates (20-50 μg) were separated on an 8-12% SDS-PAGE. The separated proteins were transferred to a PVDF membrane (#10600023, Amersham, US). The PVDF membrane was blotted with 1% BSA buffer for 1 hr at room temperature. Incubate membrane with primary antibody in 1×TBST with 5% BSA at 4° C. overnight. Wash the membrane with 1×TBST 3 times. Incubate membrane with HRP-conjugated secondary antibody for 1h at room temperature. Wash the membrane with 1×TBST 3 times. The signals were visualized with super ECL plus (#36208ES76, Yeasen Biotech, China) and image system (Azure C300, Azure Biosystems, US).

### In Vivo Efficacy in Orthotopic Models

In vivo efficacy was evaluated using the syngeneic mouse model derived from BaF3 cells with T315I and/or compound mutations. Tumor models were established by intravenously injecting tumor cells (1×10⁵/mouse) into the caudal vein under sterile conditions. Mice were randomized into control and treatment groups with 8-10 mice per group and start the treatment from the second day post inoculation. The animal body weights were measured twice a week. The antitumor activity curves of test articles were plotted with the treatment time (day) on the X-axis, and corresponding survival rate on the Y-axis. A Log-rank (Mental-cox) test was employed to analyze the statistical significance of any difference between the treatment group and the control group. Prism version 7 (GraphPad Software Inc., San Diego, CA) was used for all statistical analysis and for graphic presentation.

### Results

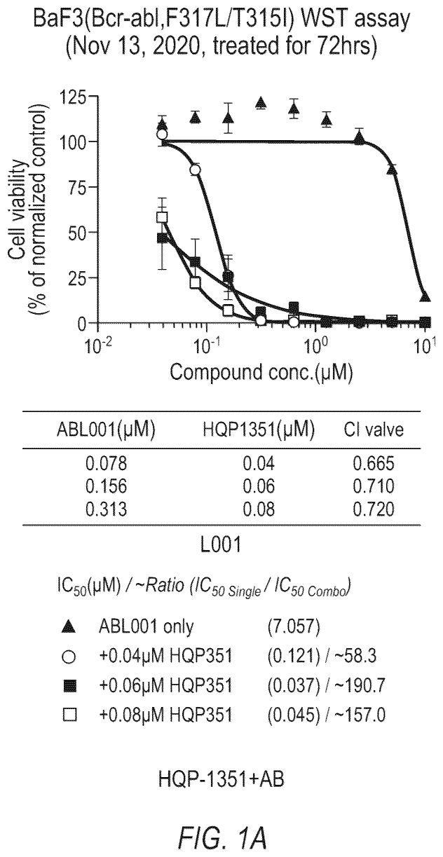

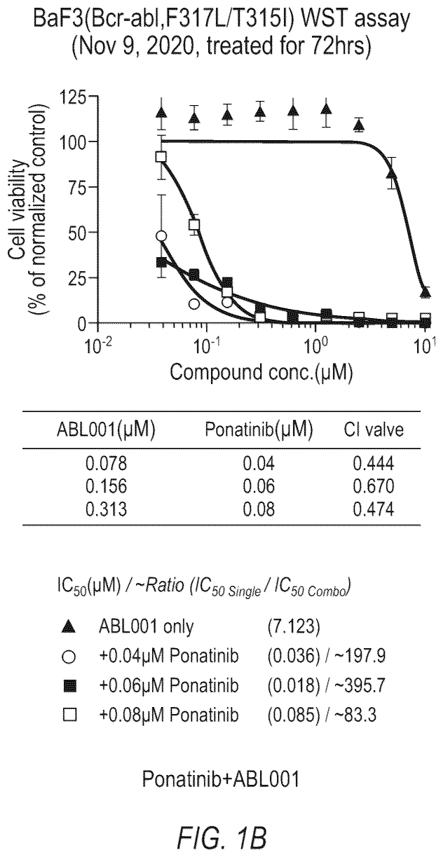

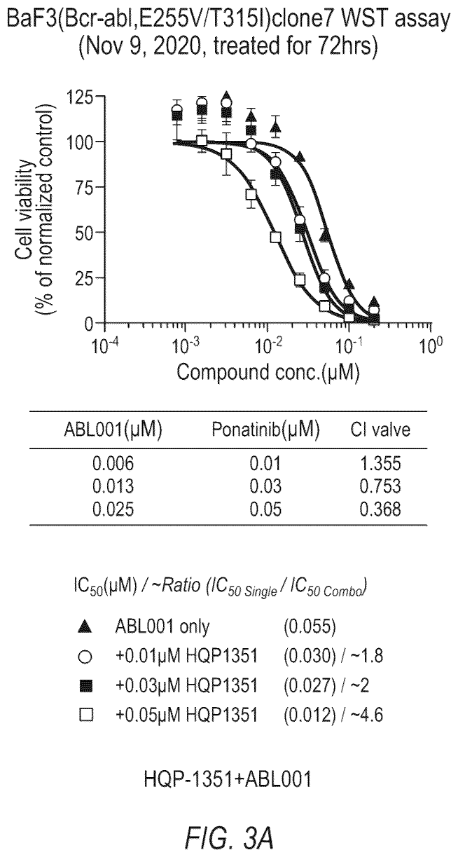

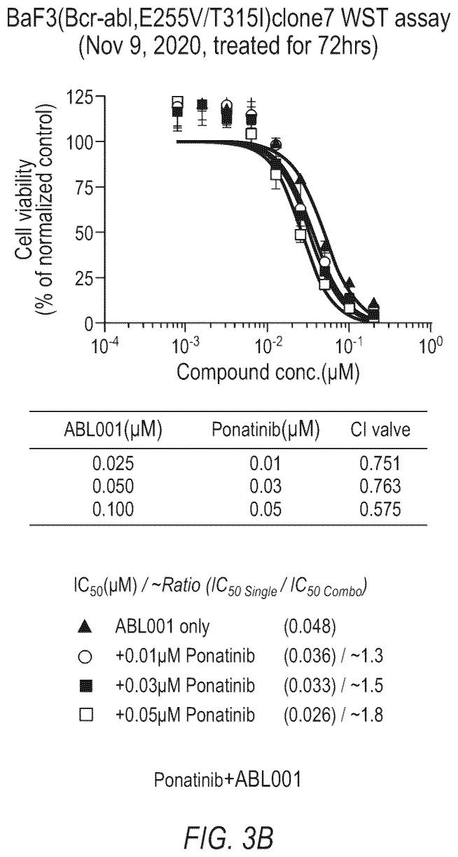

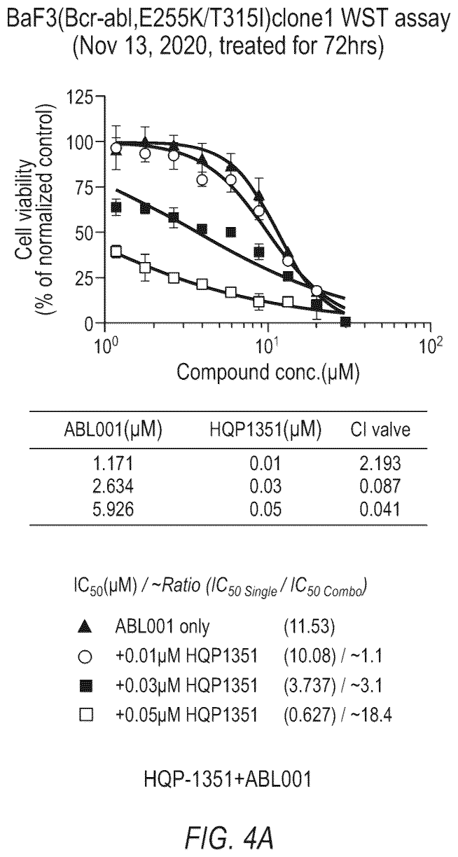

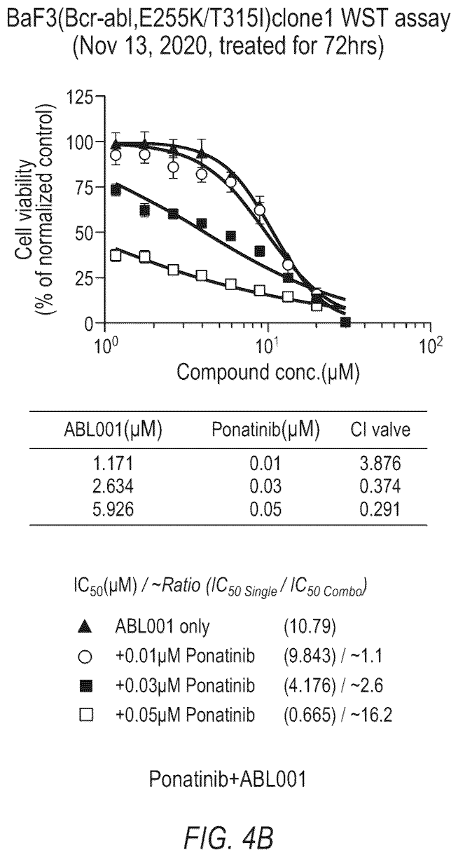

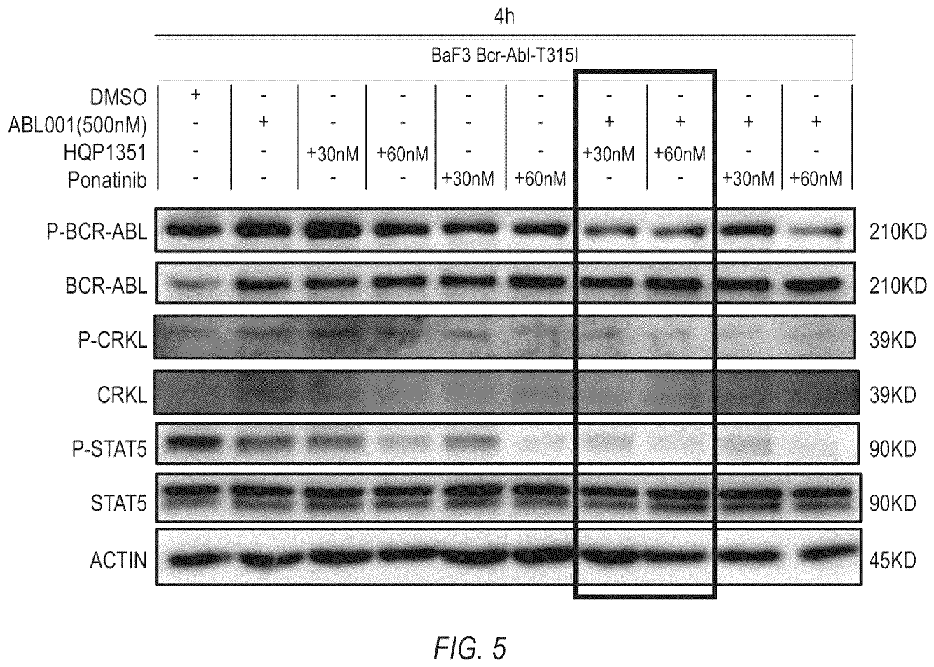

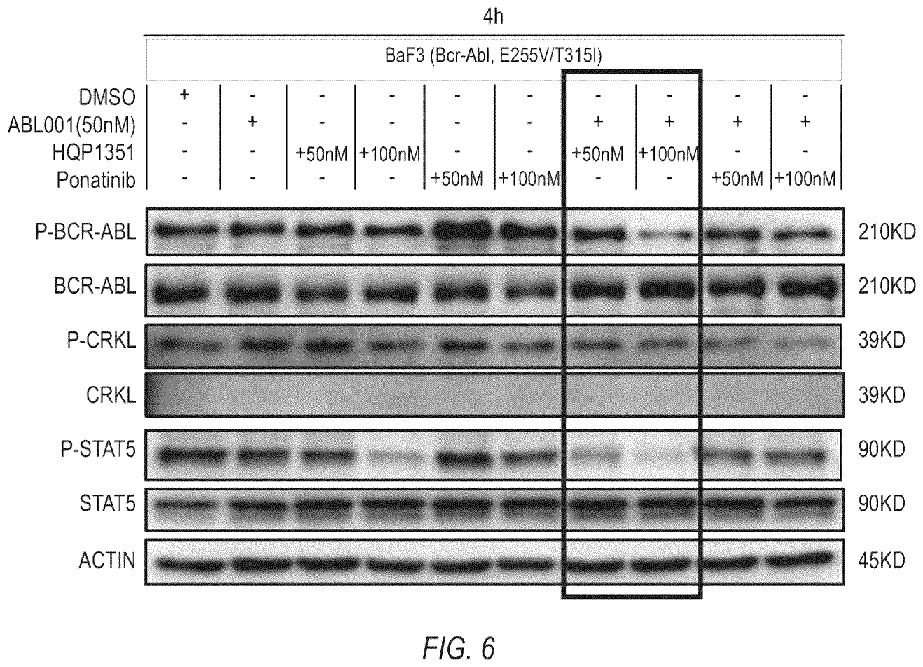

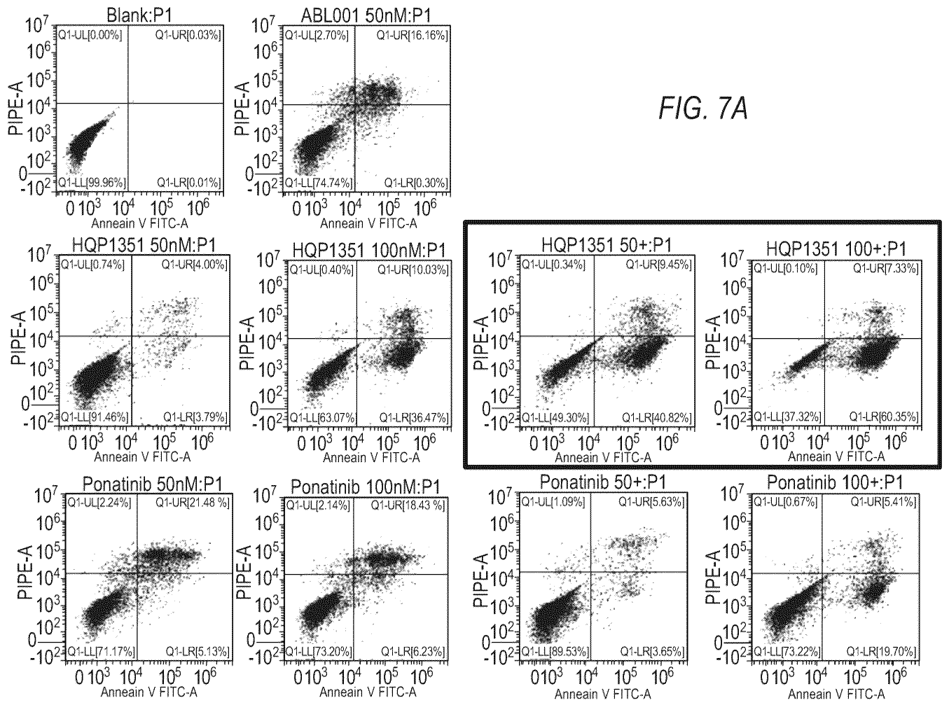

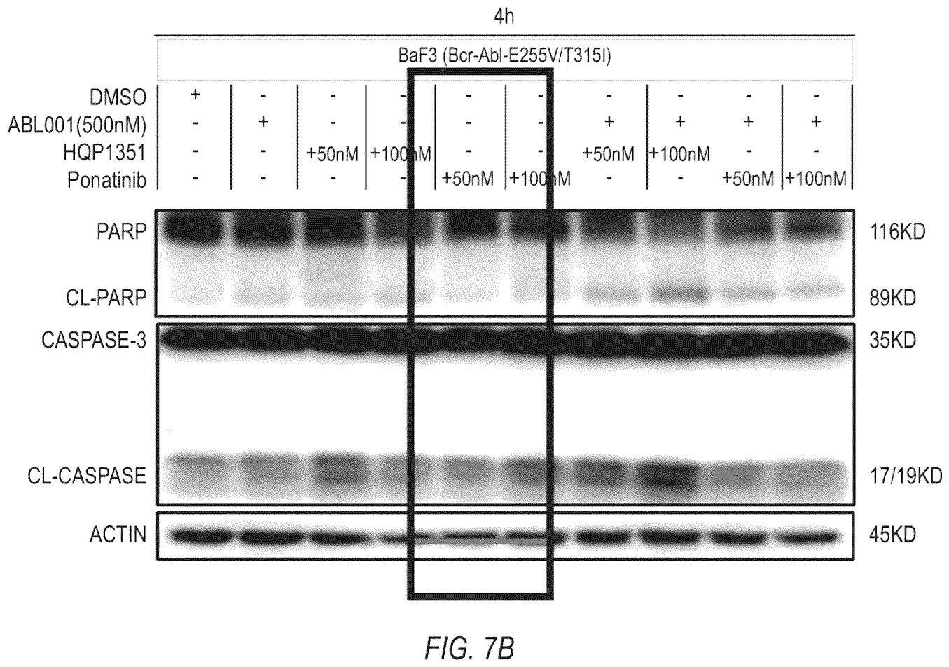

A series of cell lines expressing BCR-ABL single or compound mutations were constructed based on BaF3 murine pro-B cell line. Effect of HQP-1351 as a single agent or in combination with asciminib were analyzed using anti-proliferation assay, Western blot and FACS assays in vitro. The cell based anti-proliferation studies demonstrated superior activity of HQP-1351 toward BCR-ABL single or compound mutations with IC₅₀ values ranging between 6-300 nM. In particular, HQP-1351 was more effective than ponatinib, asciminib and other TKIs against those compound mutations. Moreover, the combination of HQP-1351 with asciminib was highly effective against BCR-ABL compound mutations, especially those containing T315I. In vivo studies further revealed that co-administration of HQP-1351 with asciminib (ABL001) resulted in significant prolongation of survival compared to single agents. Importantly, the anti-tumor effect was more potent than that of ponatinib plus asciminib in models harboring T315I or compound mutations. The results are shown in Tables 1-6, and FIGS. 1-11.

TABLE 1
HQP-1351: In vitro Single effect of Bcr-Abli in BaF3(Bcr-Abl) cell lines

| Mutated | BaF3(Bcr-Abl) | Anti-proliferation (IC50, nM) | | | | | |
|---|---|---|---|---|---|---|---|
| region | Mutant Cell | Imatinib | Nilotinib | Dasatinib | Ponatinib | HQP1351 | ABL001 |
| Wild-type | / | 565 ± 656 | 31 ± 4 | 10 + 3 | 11 | 6 + 3 | 31 ± 4 |
| SH2-contact | M351T | 1298 ± 542 | 37 ± 4 | 8 ± 4 | 13 ± 1 | 9 ± 1 | 47 ± 34 |
| region | | | | | | | |
| Substrate- | F359V | >1000 | 1710 ± 635 | 598 ± 624 | 466 ± 73 | 50 ± 16 | 6066 ± 355 |
| binding | | | | | | | |
| region | | | | | | | |
| P-loop | E255K | 8222 ± 484 | 648 ± 395 | 14 ± 1 | 49 ± 4 | 22 ± 13 | 10 |
| | 7253H | 8936 ± 1774 | 497 ± 122 | 11 ± 2 | 37 ± 4 | 7 ± 1 | 28 ± 13 |
| | E255V | 7565 ± 3268 | 587 ± 151 | 29 ± 15 | 56 ± 1 | 27 ± 11 | 24 ± 4 |
| | M244V | 2963 ± 83 | 236 ± 152 | 40 ± 1 | 75 ± 42 | 41 ± 8 | 5223 ± 4899 |
| | G250E | 6032 ± 1773 | 344 ± 18 | 28 ± 7 | 13 ± 1 | 29 ± 4 | 26 ± 16 |
| Gate keeper | T3151 | >10000 | 3425 ± 650 | 2525 ± 322 | 33 ± 11 | 24 ± 10 | 148 ± 14 |
| Hinge region | F317L | 526 ± 56 | 89 ± 8 | 11 ± 1 | 7 ± 1 | 8 ± 3 | 6 ± 3 |
| | F311I | 3547 ± 223 | 226 ± 122 | 13 ± 0 | 30 ± 8 | 23 ± 13 | 107 ± 1 |
| SH3-contact | V299L | 1987 ± 1237 | 103 ± 6 | 118 ± 2 | 10 ± 4 | 8 ± 4 | 562 ± 552 |
| Region | | | | | | | |
| Other region | E453K | 1013 ± 75 | 70 ± 45 | 12 ± 1 | 4 ± 0 | 3* | 4 ± 2 |
| | E459L | 2855* | 319* | 37* | 42* | 106* | 112* |

TABLE 2
HQP-1351: In vitro Single effect of Bcr-Abli in BaF3(Bcr-Abl) cell lines

| | BaF3(Bcr-Abl) | Anti-proliferation (IC50, nM) | | | | | |
|---|---|---|---|---|---|---|---|
| | Mutant Cell | Imatinib | Nilotinib | Dasatinib | Ponatinib | HQP1351 | ABL001 |
| T315I- | T315I-E255V | >10000 | 6467 ± 4431 | 3571 ± 1385 | 244 ± 125 | 26 ± 11 | 93 ± 86 |
| Compound | T315I-Y253H | >10000 | >10000 | 7080 ± 3233 | 889 ± 100 | 114 ± 1 | 6981 ± 2481 |
| mutation | T315I-G250E | >10000 | 8511 ± 5599 | 5001 ± 2939 | 130 ± 16 | 33 ± 2 | 8944 ± 748 |
| | T315I-255K | >10000 | >10000 | 4706 ± 803 | 339 ± 12 | 40 ± 5 | 8944 ± 748 |
| | T315I-F359V | >10000 | 4586 ± 1397 | 3392 ± 211 | 101 ± 22 | 20 ± 10 | 6631 ± 1201 |
| | T315I-F317L | >10000 | >10000 | >10000 | 688 ± 412 | 117 ± 23 | 860 ± 96 |
| | T35I-F311I | 7144 ± 2459 | >10000 | 4789 ± 1739 | 438 ± 88 | 78 ± 46 | 7061 ± 1423 |
| | T315I-H396R | 8953 ± 5314 | >10000 | 9286 ± 3386 | 211 ± 134 | 79 ± 54 | >10000 |
| | T315I-M351T | 7603 ± 1498 | >10000 | 7683 ± 3645 | 127 ± 5 | 67 ± 44 | >10000 |
| | T315I-E453K | 8466 ± 1628 | >10000 | 4724 ± 155 | 130 ± 5 | 61 ± 27 | 2936 ± 74 |
| | T315I-E459K | >1000000 | >100000 | 4869 ± 702 | 104 ± 1 | 109 ± 4 | 6001 ± 833 |
| | T316I-M244V | >100000 | >100000 | 3067 ± 904 | 136 ± 13 | 76 ± 53 | 7242 ± 211 |
| Non-T315I- | T315M | >10000 | >10000 | >10000 | 1987 ± 1414 | 217 ± 131 | 996 ± 405 |
| Compoound | Y253H-E255V | >10000 | 7026 ± 2183 | 231 ± 92 | 772 ± 220 | 122 | 5014 ± 2920 |
| mutation | F317L-F359V | 7195 ± 1729 | 926 ± 24 | 50 ± 12 | 24 ± 12 | 25 ± 13 | 5214 ± 810 |
| | Y253H-F359V | >10000 | >10000 | 110 ± 1 | 432 ± 23 | 311 ± 35 | >10000 |
| | G250E-V299L | 6486 ± 2622 | 641 ± 368 | 570 ± 559 | 12 ± 3 | 14 ± 2 | 2601 ± 2903 |
| | F317L-M351T | 3367 ± 1685 | 263 ± 134 | 100 ± 30 | 65 ± 28 | 32 ± 25 | 5401 ± 6188 |
| | V299L-F39V | 1898 ± 110 | 1005 ± 43 | 122 ± 3 | 115 ± 11 | 32 ± 4 | 4933 ± 1617 |

TABLE 3
Superior effect when HQP-1351 is combined with
ABL001 in T315I orthotopic tumor model.

| | | | Survival |
|---|---|---|---|
| | Survive/Total | | prolongation |
| Group | @ D43 | Median survival | (%) |
| Vehicle | 0/10 | 21.0 | — |
| ABL001, 60 mg/kg | 0/10 | 24.5**** | 16.67 |
| Ponatinib, 20 mg/kg | 0/10 | 27.0**** | 28.57 |
| HQP1351, 20 mg/kg | 0/10 | 27.0**** | 28.57 |
| Ponatinib + ABL001 | 0/10 | 29.0****^{####$$} | 38.1 |
| HQP1351 + ABL001 | 0/10 | 35.0****^{####$$$$} | 66.67 |

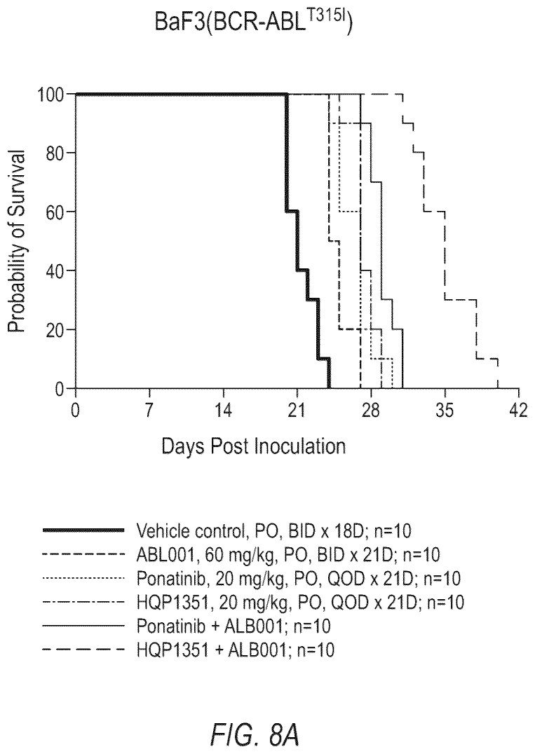

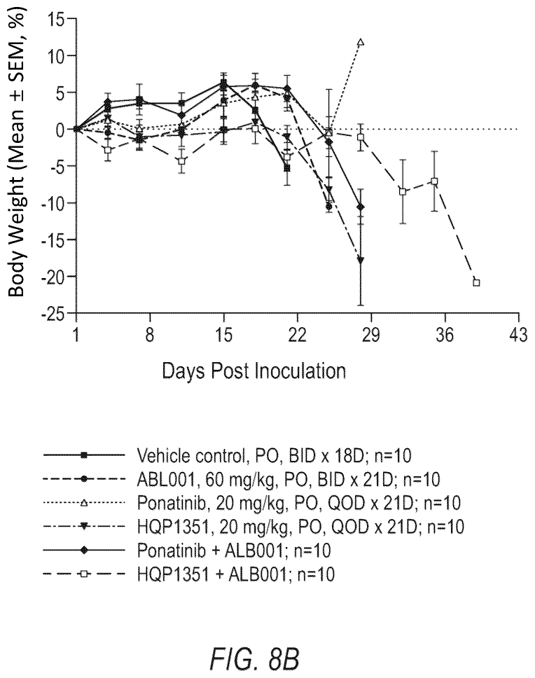

****P < 0.0001;
***P < 0.0002;
**P < 0.0021 vs. Vehicle Control;
^{####}P < 0.0001;
^{###}P < 0.0002;
^{#}P < 0.0332 vs. ABL001;
^{$$$$}< 0.0001;
^{$$}P < 0.0021 vs. Ponatinib or HQP1351
The result of the study also shown in FIGS. 8A and 8B.

TABLE 4
Superior effect when HQP-1351 is combined with
ABL001 in Y253H/T315I orthotopic tumor model.

| | Survive/ | | Survival |
|---|---|---|---|
| | Total | | prolongation |
| Group | @ 24 | Median survival | (%) |
| Vehicle control | 0/8 | 17.0 | |
| ABL001, | 0/8 | 21.0*** | 23.53 |
| 60 mg/kg | | | |
| Ponatinib, | 0/8 | 20.0** | 17.65 |
| 20 mg/kg | | | |
| HQP1351, | 0/8 | 18.5 | 8.82 |
| 20 mg/kg | | | |
| Ponatinib + | 0/8 | 22.0****^{$###} | 29.41 |
| ABL001 | | | |
| HQP1351 + | 0/8 | 24.0****^{$$$$####} | 41.18 |
| ABL001 | | | |

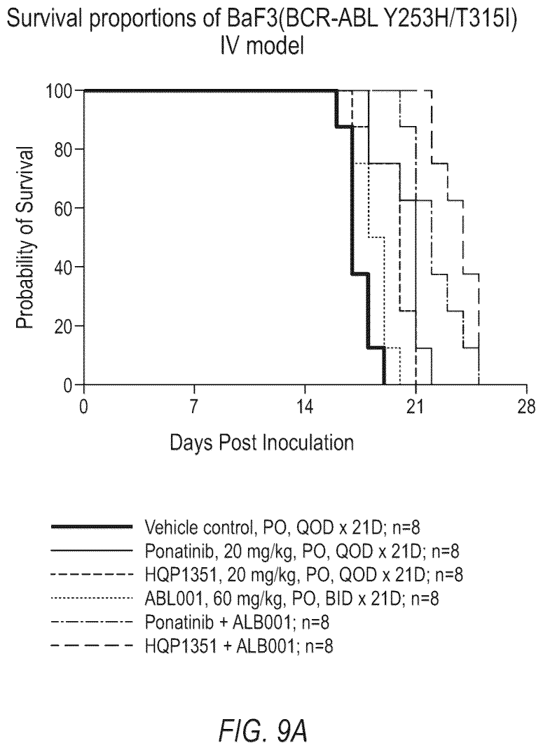

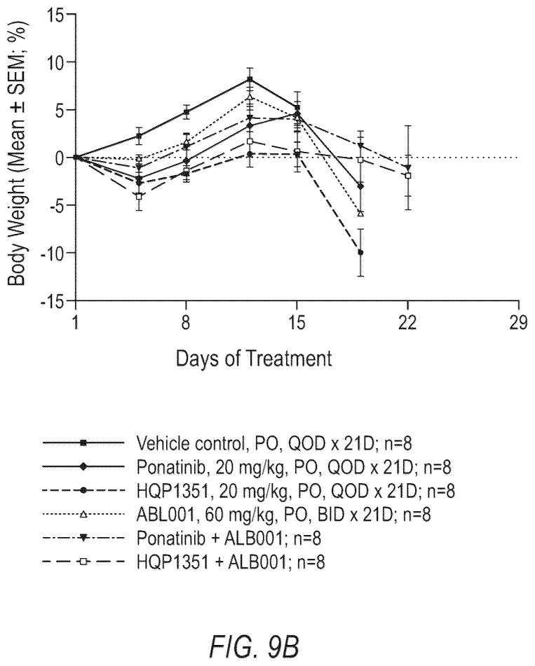

****P < 0.0001 vs. Vehicle Control;
^{$$$$}P < 0.0001;
^{$}P < 0.0332 vs. Ponatinib or HQP1351
^{####}P < 0.0001;
^{###}P < 0.0002 vs. ABL001
The result of the study also shown in FIGS. 9A and 9B.

TABLE 5
Superior efficacy of HQP-1351 and ALB001
in F317L/T315I orthotopic tumor model

| | Survive/ | | Survival |
|---|---|---|---|
| | Total | | prolongation |
| Group | @ 24 | Median survival | (%) |
| Vehicle control | 0/8 | 17.5 | |
| Ponatinib, 20 mg/kg | 1/8 | 27.0**** | 54.29 |
| HQP1351, 20 mg/kg | 0/8 | 25.0****^{$} | 42.86 |
| ABL001, 60 mg/kg | 0/8 | 18.0 | 2.86 |
| Ponatinib + | 6/8 | Undefined****^{$####} | |
| ABL001 | | | |
| HQP1351 + | 8/8 | Undefined****^{$$$$####} | |
| ABL001 | | | |

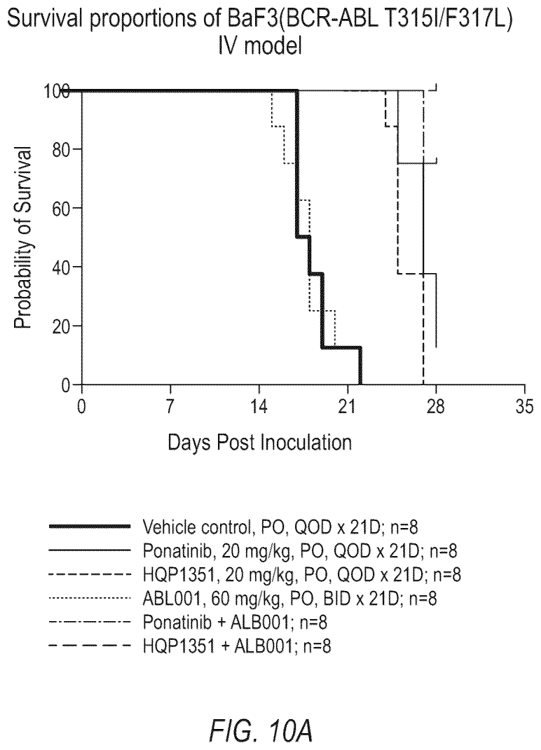

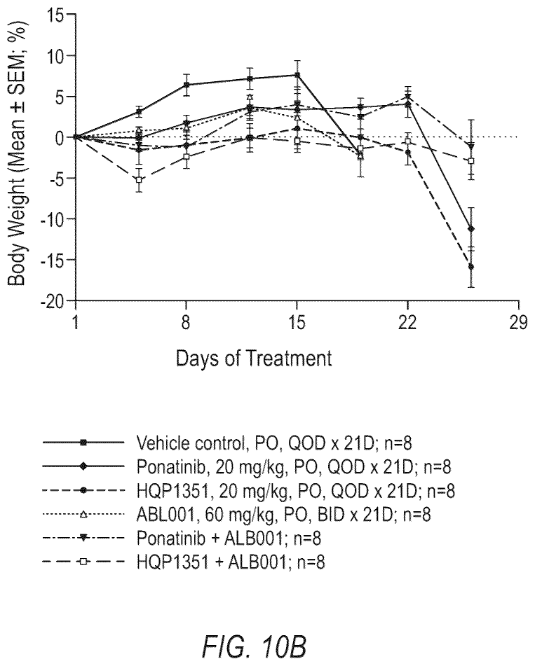

****P < 0.0001 vs. Vehicle Control;
^{$$$$}P < 0.0001;
^{$}P < 0.0332 vs. Ponatinib or HQP1351
^{####}P < 0.0001 vs. ABL001
The result of the study also shown in FIGS. 10A and 10B.

TABLE 6
Comparable efficacy of HQP-1351 and ALB001
in E255V/T315I orthotopic tumor model

| | Survive/ | | Survival |
|---|---|---|---|
| | Total | | prolongation |
| Group | @ D26 | Median survival | (%) |
| Vehicle | 0/10 | 21.0 | — |
| Ponatinib, 20 mg/kg | 0/10 | 24.5**** | 16.67 |
| HQP1351, 20 mg/kg | 0/10 | 27.0**** | 28.57 |
| ABL001, 60 mg/kg | 0/10 | 27.0**** | 28.57 |
| Ponatinib + ABL001 | 0/10 | 29.0****^{####$$} | 38.1 |
| HQP1351 + ABL001 | 0/10 | 35.0****^{####$$$$} | 66.67 |

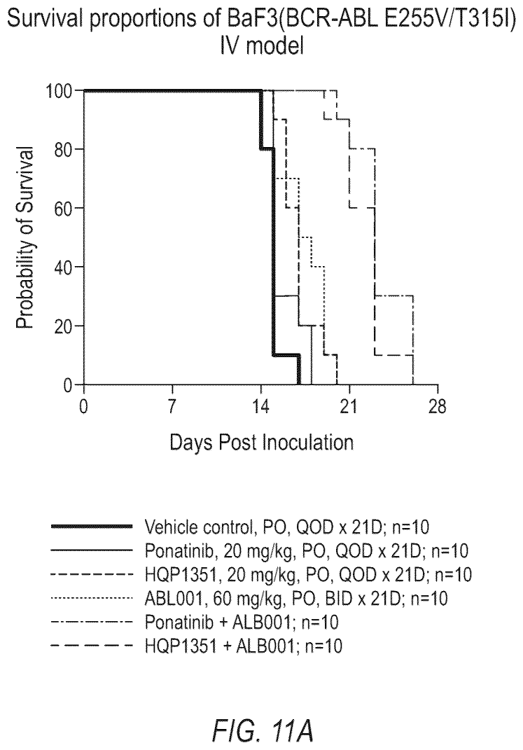

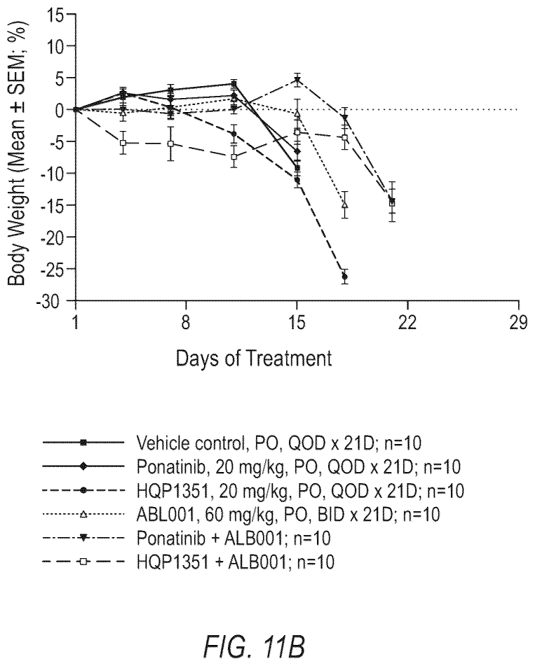

****P < 0.0002;
**P < 0.0021 vs. Vehicle Control;
^{$$$}P < 0.0002 vs. Ponatinib or HQP1351
^{###}P < 0.0002; vs. ABL001
The result of the study also shown in FIGS. 11A and 11B.

### Conclusion

The results demonstrated that the combination of ATP binding site inhibitor olverembatinib and allosteric inhibitor have synergistic anti-tumor effect on tumor cells harboring single or compound mutations in BCR-ABL. Without being binding by the theory, the combined treatment synergistically downregulated phosphorylation of BCR-ABL and the downstream proteins CRKL and STAT5, and augmented cleavage of Caspase-3 and PARP-1, thus triggered apoptosis and subsequently enhanced the antitumor effects. This novel strategy may help to overcome the secondary compound mutations post the treatment with TKIs.

### Example 2 Study of HQP-1351 as a SRC Inhibitor that Increases the Antitumor Immunity in Non-Small Lung Cancer Study

### General Method

A cytotoxicity assay was used to determined antitumor activities of HQP-1351 in NSCLC cell lines. Furthermore, the expression of PD-L1 in HQP-1351 treated cells was investigated by western blots, quantitative PCR and flow cytometry. In addition, the effects of HQP-1351 alone or in combination with ICIs in enhancing antitumor immunity were also determined in vitro and in vivo. Gene manipulation technique was used to establish stable SRC knockdown NSCLC cells to explore the underlying mechanisms.

### Cell Culture and Regents

NSCLC cell lines (A549, H1299, H460), Lewis lung cancer (LLC) cell line, and 293 T cell line were obtained from the American Type Culture Collection (ATCC, USA) and validated by short-tandem-repeat (STR) analysis. Cells were cultured in either RPMI-1640 (for NSCLC cell lines) or DMEM (for LLC cells and 293 T cells) containing 10% fetal bovine serum and maintained at 37° C. in a humidified 5% C02 incubator. Peripheral blood mononuclear cells (PBMCs) were cultured in T cell medium (RPMI-1640 supplemented with 10% human serum, 5% L-glutamine-penicillin-streptomycin solution (Sigma-Aldrich, USA), and IL-2 (100 IU/mL). HQP-1351 was provided by Ascentage Pharma Group Inc. For in vitro studies, HQP-1351 was dissolved in DMSO at a concentration of 10 μM and stored at −20° C. For in vivo studies, HQP-1351 was dissolved in 0.2% HPMC.

### Cell Viability Assays

Cells were cultured at a density of 3×10³ cells per well containing 200 μl of culturing medium in 96-well plates for 12 hours. After adherence, cells were pretreated with the indicated concentration of HQP-1351 for 72 hours. 20 μl CCK8 reagents (Dojindo Laboratories, Japan) were added to 200 ul culture medium per well and incubated for 2-4 h at 37° C. Then the absorbance value was measured with a spectrophotometer at 450 nm. All experiments were performed in 3 replicates per trial and conducted at least three times. The dose-response curves and half maximal inhibitory concentration (IC50) values were analyzed with nonlinear regression on GraphPad Prism version 8.0.

### Western Blot Analysis

Cells were treated with the indicated concentrations and washed twice with cold PBS. Whole cell extracts were collected in RIPA lysis buffer (Santa Cruz Biotechnology, Germany), and protein concentration of the lysates was measured using a BCA Protein Assay Kit (ThermoFisher, USA). The protein samples were electrophoresed through a 10% SDS-PAGE gel and transferred to a polyvinylidene difluoride (PVDF) membrane (Roche, USA). After blocking, membranes were probed with primary antibodies (1:1000) against SRC, p-SRC, PD-L1, GAPDH, 0-tubulin (Cell Signaling Technology, USA) followed by washing and incubation with a secondary antibody (1:5000) conjugated to horseradish peroxidase (Santa Cruz Biotechnology, USA). Protein bands were visualized by applying a chemiluminescent reagent (Pierce ECL kit, Thermo Fisher Scientific, USA) and protein level were quantified by Image Lab (Bio-Rad Laboratory, USA).

### RNA Extraction and Quantitative Real-Time PCR

Total cellular RNA was extracted by Trizol (Invitrogen, USA) for mRNA analysis. RNA was reverse-transcribed to cDNA profile through using the Transcriptor First Strand cDNA Synthesis kit (Roche Applied Science, USA) and then Real time PCR reactions were performed using the FastStart SYBR Green Master (ROX) reagent (Roche Applied Science, USA) according to the manufacturer's instructions. Real-time PCR analyses were conducted using the Biorad CFX96 system with SYBR green (Bio-Rad, USA) and the appropriate primers to estimate the mRNA expression level of SRC. Data were normalized to GAPDH levels in the samples in triplicates. The primes are as follows:

SRC forward:
GAGCGGCTCCAGATTGTCAA;
reverse:
CTGGGGATGTAGCCTGTCTGT;
PDL1 forward:
TATGGTGGTGCCGACTACAA;
reverse:
TGCTTGTCCAGATGACTTCG;
GAPDH forward:
GGTGAAGGTCGGAGTCAACGG;
reverse:
CCTGGAAGATGGTGATGGGATT.

Transfection of shRNA and Plasmid DNA

SRC shRNAs and a shRNA scramble control (GeneCopoeia, USA) were transiently transfected along with a pSIH-H1-puro Lentivector Packaging Kit (System Biosciences, USA). Transfections were carried out in 293 T cells grown to ˜80% confluency in 10 cm dishes using Lipofectamine 2000 transfection reagent (Life Technologies, USA) and following the manufacturer's instructions. H460 and H1299 cells were infected and incubated with the viral particles overnight at 37° C. At 48 h after transfection, cells were placed under puromycin selection by supplementing the growth medium with puromycin (1.5 μg/ml for H460, and 2 μg/ml for H1299). Stable repression of gene expression was verified by Western blotting and RT-PCR.

### Colony Formation Assay

As effector cells, human PBMCs were purified from the blood of healthy volunteers using Ficoll gradient centrifugation (Solarbio, Beijing). The purity of the isolated cells was >95%, as determined in flow cytometry (FCM). NSCLC cell lines were seeded in a 96-well plate treated or not treated with HQP-1351. Human peripheral blood mononuclear cells were activated with 100 ng/ml CD3 antibody, 100 ng/ml CD28 antibody and 10 ng/ml IL-2 and then co-cultured with NSCLC cells. Cells were treated with PD-L1 Ab or not and co-cultured with activated PBMCs at several target-to-effector ratios (1:0, 1:1, 1:4, 1:16) (all samples in triplicate). After 4 days of co-incubation, 24-well plates wells were washed with PBS twice to remove PBMCs and then the survived tumor cells were fixed and stained with Giemsa staining solution. The dried plates were scanned and quantified the intensity.

### Flow Cytometry Analysis

Activated PBMCs were plated at a density of 1×10⁶/well in 6-well plates and then co-cultured with tumor cells pre-treated with HQP-1351 at 4:1 ratio for 24 h. Anti-human PD-L1 antibody, atezolizumab (Selleck Chemicals, USA) (50 μg/ml), was added to the appropriate wells. After co-culturing, the PBMCs were isolated and stained with anti-CD3 and anti-CD8 antibodies to estimate the CD8+ cell proportions. For IFN-γ, TNF-α and granzyme B analysis, PBMCs were harvested and then treated with brefeldin A (Biolegend, USA) at 37° C. for an additional 3 h to prevent extracellular secretion. Subsequently, PBMCs were fixed and permeabilized with the Intracellular Fixation and Permeabilization Buffer Set Kit (eBioscience, USA) following the manufacturer's instructions. Then percentages of IFN-γ, TNF or Granzyme B positive cells in CD8+ T cells were labeled via intracellular staining and detected by flow cytometry. Antibodies for flow cytometry analysis were purchased from eBiosciences, USA. Matched isotype controls were used for each antibody to determine gates. FlowJo (Treestar, USA) software was used for the analysis of flow cytometry data. Standardized fluorescence intensities were calculated by dividing the median fluorescence intensities of specific antibodies by the median fluorescence intensities of isotype controls. The results are expressed as mean±SD of three independent experiments.

### In Vivo Mouse Studies

C57BL/6 mice were obtained from from Vital River Laboratory Animal Technology Co., Ltd, (Beijing, China), and kept in a specific pathogen-free (SPF) barrier facility at the Animal Center of Sun Yat-sen University Cancer Center. The female mice with 8-12 weeks old were used for all animal experiments. Experiments were approved by the institutional committee of Sun Yat-sen University Cancer Center, and conducted in accordance with protocols approved by the Guangdong Provincial Animal Care and Use Committee.

LLC cells (10×10⁵ cells in 200 μL growth medium) were subcutaneously injected into the right flank of immunocompetent C57BL/6 mice. Tumor growth was measured with calipers every 3 days and the tumor volumes were calculated by applying the following formula: ½(length×width2). When tumors reached approximately 100 mm3, mice were randomized into control or experimental groups. A terminal event was defined as tumors reaching a size of 2000 mm3, at which point animals were euthanized.

Mice were treated with HQP-1351 or rat anti-PD-L1 antibody (αPD-L1, clone 10F.9G2; BioLegend, USA) alone, the combination of HQP-1351 and αPD-L1, or saline and IgG2bx (clone RTK4530; BioLegend, USA). HQP-1351 (50 mg/kg) was administered via gavage from day 13, every day, after tumor implantation. Anti-PD-L1 antibody therapy (10 mg/kg) was administered intraperitoneally weekly on days 16, 23, 30, 37, and 44. Survival analysis was performed using Kaplan-Meier analysis and log-rank test.

### Statistical Analysis

Statistical analysis was carried out using IBM SPSS Statistics 19 software or GraphPad Prism using Student's t-test or one-way ANOVA or Dunnett's test. All experiments were repeated in triplicate. Data are expressed as mean±standard deviation (SD). Statistical significance was defined as P<0.05.

### Results

### HQP-1351 Potentiates Anti-PD-L1 Efficacy In Vitro

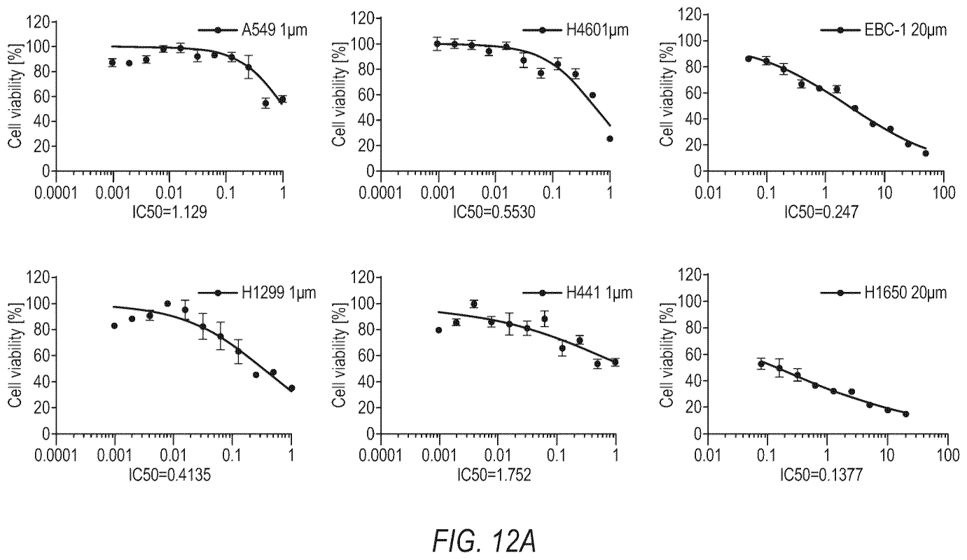

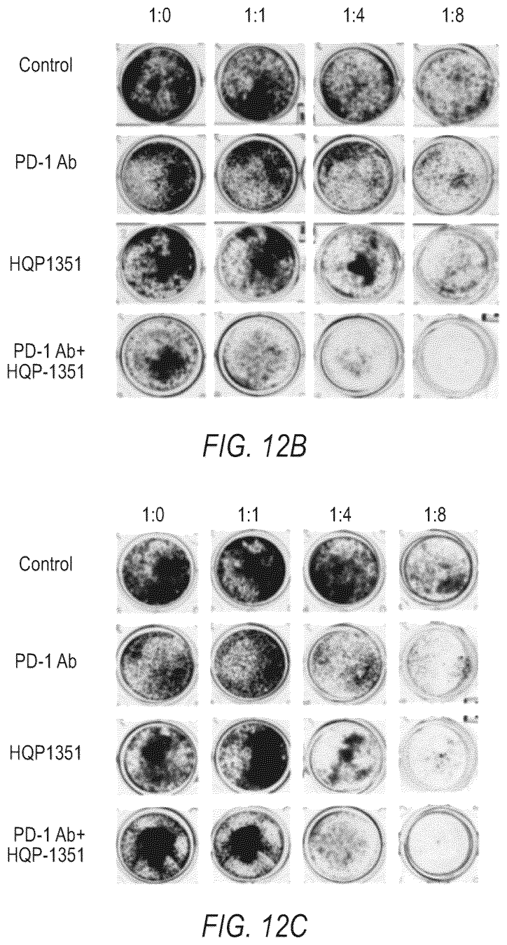

First, in order to exclude any underlying bias caused by variation in growth suppression induced by the HQP-1351, we performed growth inhibition curves for different cell lines and established an inhibitory concentration of 50% (IC50) (FIG. 1a). Then, to investigate whether HQP-1351 combined with PD-1/PD-L1 blockade can exert a synergistic immunotherapeutic effect, we tested the efficacy of the combined use of HQP-1351 and anti-PD-L1 blocking antibodies in vitro. HQP-1351 combined with PD-L1 antibody (atezolizumab) showed significantly higher tumor growth inhibition compared to HQP-1351 alone or PD-L1 blockade alone. FIGS. 12A-C show that HQP-1351 potentiates anti-PD-L1 efficacy in vitro. The Cytotoxicity of HQP-1351 on different human cancer cells were determined by CCK8 as described in materials and methods. Each point represents the mean±standard deviations (SDs) of three independent experiments performed. (B-C). T cell cytotoxicity test by colony formation assay. The survival of HQP-1351 pretreated H460 and H1299 cells, un-pre-treated cells, treated with PD-1 Ab or without, and co-cultured with PBMCs (targeted cells: effector cells=1:0, 1:1, 1:4, 1:8) in 24-well plates for 4 days was estimated.

### HQP-1351 Potentiates Anti-PD-L1 Efficacy In Vivo

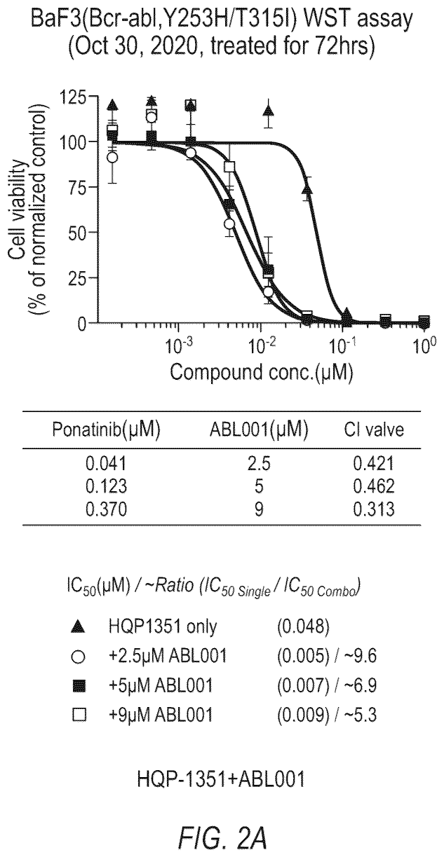

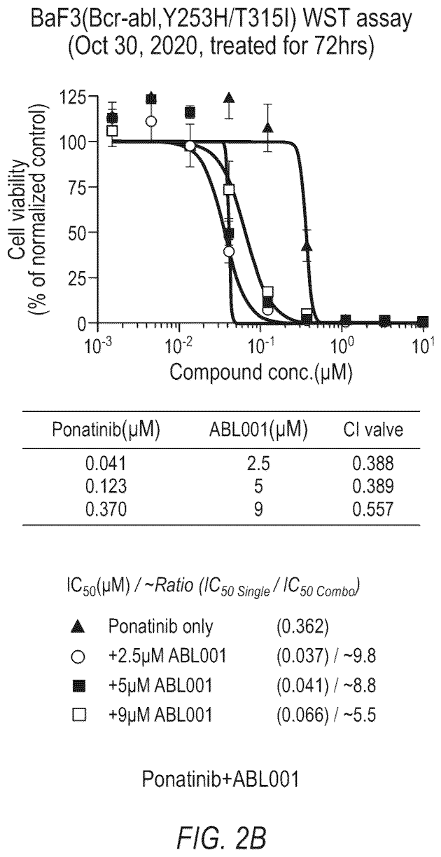

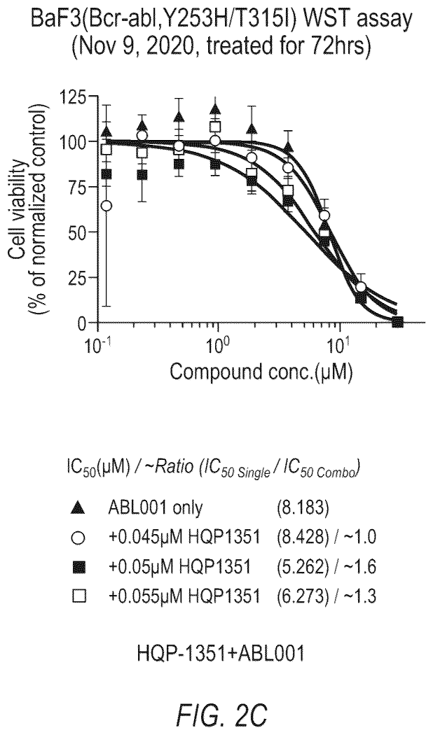

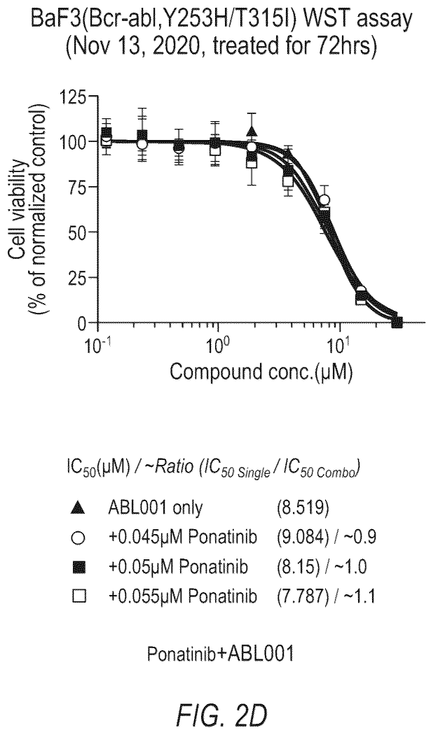

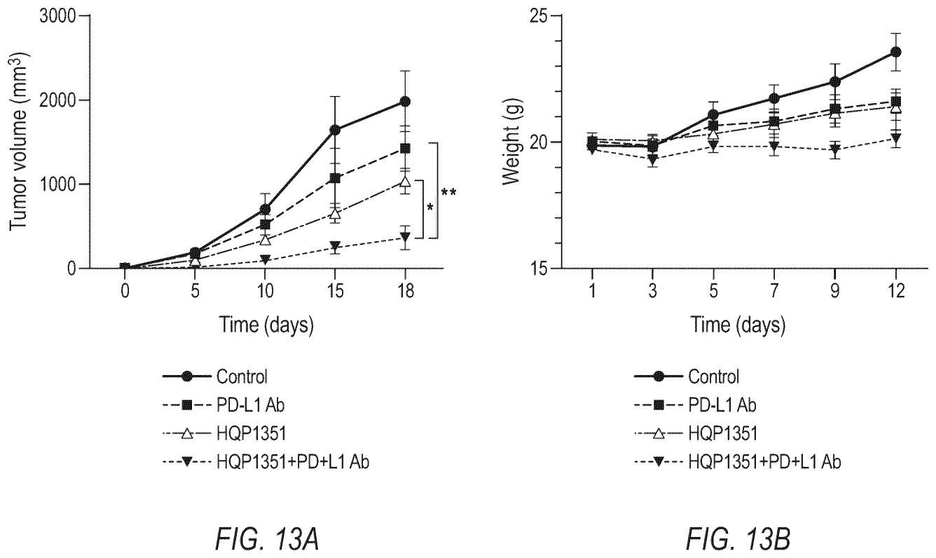

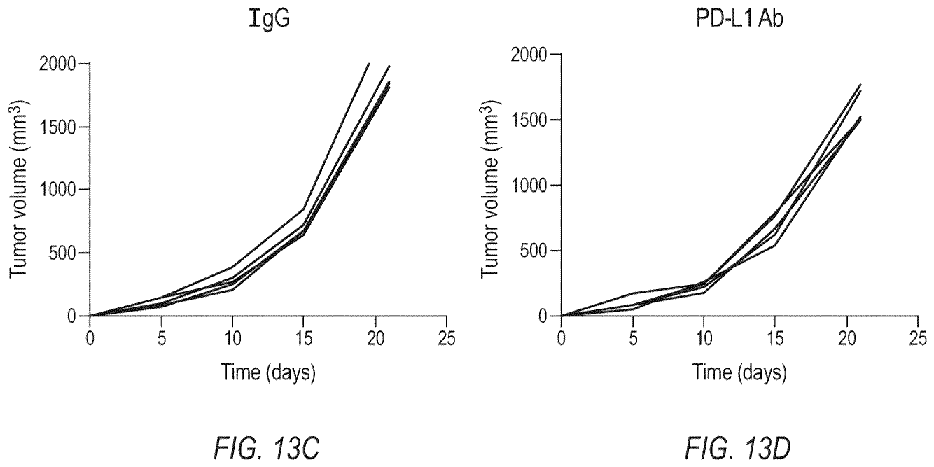

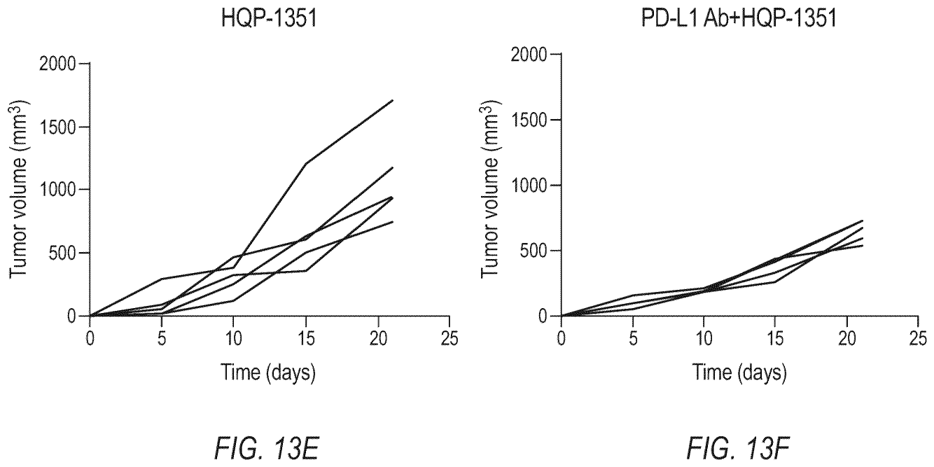

In LLC cell tumor-bearing mice, mice receiving HQP-1351 plus PD-L1 Ab treatment showed a more significant delay in tumor growth (FIG. 2a-c) compared to those receiving monotherapy with HQP-1351 or PD-L1 Ab. Meanwhile, there was no significant loss in body weight in the experimental mice suggested the combination regimen was relatively well-tolerated by the mice. FIGS. 13A-F show that HQP-1351 potentiates anti-PD-L1 efficacy in vivo. Tumor volumes were determined at the indicated days with different treatments in C57BL/6 mice (n=5). Error bars represent SEM of three independent experiments. (B) The body weight changes of C57BL/6 mice with different treatments (n=5).

HQP-1351 Suppresses p-STAT3 and PD-L1 Expression in a Dose- and Time-Dependent Manner

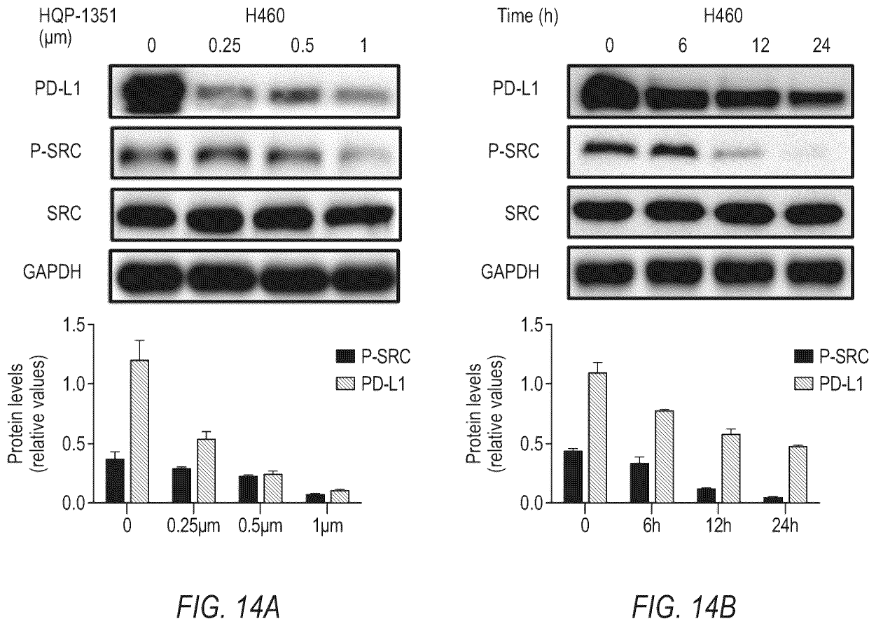

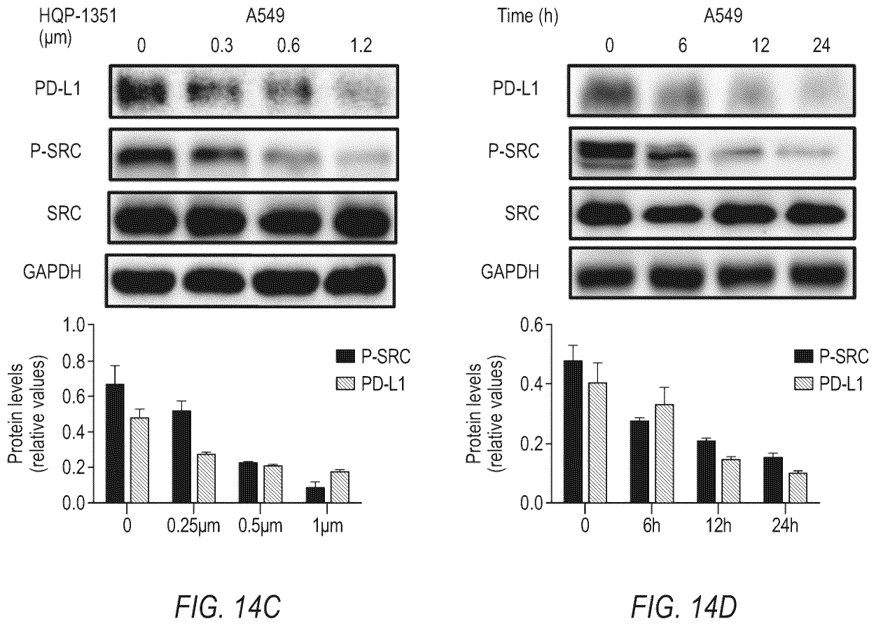

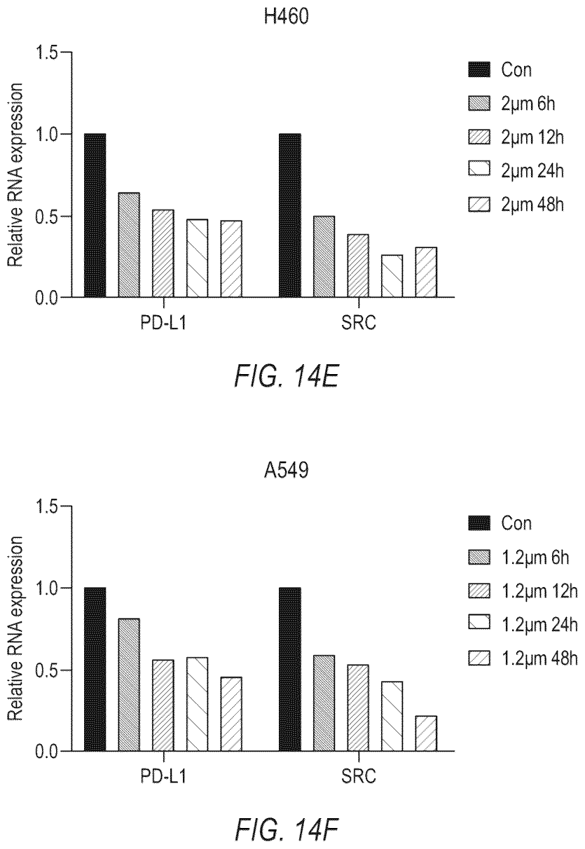

To further explore the potential mechanism of enhancement of PD-L1 antibody by HQP-1351, we further validated the inhibitory effect of HQP-1351 on PD-L1 expression. After treatment with differing concentrations of HQP-1351, we observed that HQP-1351 decreased PD-L1 expression as well as p-SRC phosphorylation in a concentration-dependent manner in NSCLC cell lines (FIG. 14A). Moreover, cells treated with 2 μM HQP-1351 at different time points showed a time-dependent suppression of PD-L1 and p-SRC levels (FIG. 14B). To test this, we also performed real-time PCR (RT-PCR) analysis (FIG. 14C). These results demonstrated that HQP-1351 downregulated expression of p-SRC and PD-L1 in a time- and concentration-dependent manner. FIGS. 14A-C show that HQP-1351 suppress p-SRC and PD-L1 expression in a dose- and time-dependent manner. In FIG. 14A, H460 and A549 cells were treated with different concentrations of HQP-1351 for 24 h, p-SRC, SRC and PD-L1 expression was measured by Western blot. In FIG. 14B, H460 and A549 cells were treated with 2 μM HQP-1351 for different time intervals, p-SRC, SRC and PD-L1 expression was measured by Western blot. In FIG. 14C H460 and A549 cells were treated with different concentrations of HQP-1351 for 24 h and treated with 2 μM HQP-1351 for different time intervals, SRC and PD-L1 expression was measured by RT-PCR.

### Conclusion

Our data demonstrated that HQP1351 act as a SRC inhibitor that enhanced T-cell-mediated anti-tumor immune responses in NSCLC. We found that the SRC inhibitor HQP-1351 robustly decreased expression of PD-L1 in NSCLC cells and animal models. The observed inhibitory effect was time- and concentration-dependent. Furthermore, our results showed that HQP-1351 and anti-PD-L1 combined to improve T-cell-mediated killing of tumor cells in vitro and in vivo, which were associated with the elevated cytokines secretion of activated CD8+T cytotoxic cells, involving IFN-γ, TNF-α and granzyme-B. Moreover, we also observed that mice treated with HQP-1351 in combination with PD-L1 blockade showed longer survival than those treated with single drug alone.

We also provide evidence that support the combination of HQP-1351 and anti-PD-1/PD-L1 as a potential combination therapeutic approach to increase the efficacy of immunotherapy in NSCLC.

### Example 3 Antitumor Activity of HQP-1351 in Philadelphia Chromosome-Positive Pre-B all Cell Line (pH+ all SUP-B15)

### Cell Viability Assay

Leukemia cells were seeded at a density of 10,000 cells in an opaque 96-well plate and incubated with increasing concentrations of HQP-1351 or with vehicle (DMSO). After treatment, cell viability was measured by Promega© Cell-Titer Glo®3D Cell Viability Assay luminescence assay kit according to instructions from the manufacturer (Promega, Madison, WI, USA, Cat #G7571). Luminescence signal was detected using a BioTek Synergy H1 Hybrid Multi-Mode (Microplate) Reader (BioTek, Shanghai). Cellular proliferative (i.e. viability) curves were plotted and anti-proliferation IC₅₀ values were calculated using Graphpad Prism version 6.0 software (GraphPad Software, San Diego, CA USA).

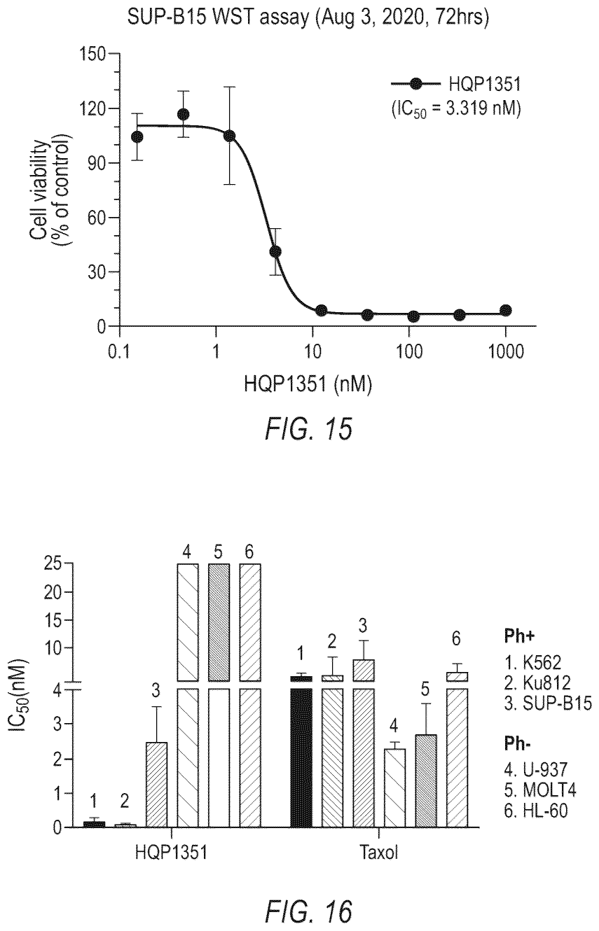

FIG. 15 shows cell viability curve of SUP-B15 cells treated with HQP1351 for 72 hours.

FIG. 16 shows the anti-proliferation IC50 values of HQP-1351 in Philadelphia chromosome positive (Ph+ or BCR-ABL1+) and negative (Ph- or BCR-ABL1-) leukemia cell lines. The data demonstrated that HQP-1351 selectively inhibited the proliferation of Ph+ cells including SUP-B15.

### Conclusion

HQP-1351 potently inhibits the proliferation of Ph+ ALL SUP-B15 cells and induces apoptosis of primary pre-B ALL cells and ALL cell lines.

### Example 4 Combination of HQP-1351 with an Anti-PD-1 Antibody in a RANCA-Derived Syngeneic Model for Treating Renal Cell Carcinoma (RCC)

HQP-1351 is a new-generation multikinase inhibitor with targets including VEGFR, fibroblast growth factor receptor (FGFR), SRC, BCR-ABL1, c-KIT, and platelet-derived growth factor receptor. HQP-1351 is currently under clinical development for relapsed or refractory chronic myeloid leukemia and gastrointestinal tumor. This study is to assess whether HQP-1351 combined with immunotherapy can promote inhibitory effects on RCC.

In cell-free kinase assays, HQP-1351 inhibited VEGFR1, -2, and -3 with IC₅₀ values of 4.2, 6.1, and 4.1 nM, respectively. Compared to lenvatinib, HQP-1351 had more potent antiproliferative effects on human umbilical vein endothelial cells. HQP-1351 also had antiproliferative activity in murine RCC lines RANCA and RAG, with IC₅₀ values of 141 and 53 nM, respectively. When olverembatinib was coadministered with an anti-PD-1 antibody (in a RANCA-derived syngeneic model, both agents exerted synergistic effects, with tumor growth inhibition rates reaching 60.6%. Mechanistically, HQP-1351 influenced cancer cell proliferation directly by inhibiting phosphorylation of FGFR and downstream proteins. Increased cleavage of caspase-3 and poly (ADP-ribose) polymerase 1 were observed, suggesting induction of apoptosis. HQP-1351 also influenced proliferation of vascular endothelial cells by inhibiting phosphorylation of VEGFR, SRC, and downstream proteins Akt and extracellular signal-regulated kinases. HQP-1351 also reduced expression of PD-L1 in RCC cells. In tumor-infiltrating lymphocyte assays, HQP-1351 increased numbers of cytotoxic T cells (CTL, CD8⁺) and natural-killer cells (NK, CD3⁻/CD49B⁺) in RANCA tumor tissues. Combined with an anti-PD-1 antibody (InVivoMab anti-mouse PD-1(CD279), purchased from Bioxcell), HQP-1351 increased CTLs, NK cells, dendritic cells (DCs, MHC-II⁺/CD11C⁺), and M1 macrophages (F4/80⁺/CD11B⁺/CD86⁺) in RANCA tumor tissues, indicating an immunoregulatory effect of olverembatinib.

Taken together, these data suggest that combining HQP-1351 with a checkpoint inhibitors confers synergistic antitumor effects in an RCC cancer mouse model by targeting tumor growth, angiogenesis, and immune regulation. This novel combination may provide an alternative approach to enhance treatment effects with CPIs in renal cancers.

### CONCLUSION

HQP-1351 enhances antitumor effects of immunotherapy in renal cell carcinoma (RCC).

We claim:

1. A method of treating cancer comprising co-administering to a subject in need thereof:
a) a compound of formula (I) or a pharmaceutically acceptable salt thereof; and
b) an allosteric inhibitor;
wherein formula (I) has the following structure: 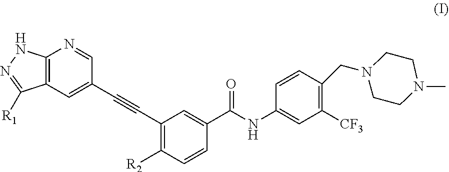
wherein
R₁ is hydrogen, C₁₋₄ alkyl, C₃₋₆ cycloalkyl, C₁₋₄ alkyloxy, or phenyl; and
R₂ is hydrogen, C₁₋₄ alkyl, C₃₋₆ cycloalkyl, or halogen; wherein the cancer is hematological malignancy.

2. The method of claim 1, wherein the compound of formula (I) is HQP-1351 or a pharmaceutically acceptable salt thereof, wherein HQP-1351 has the following structure: 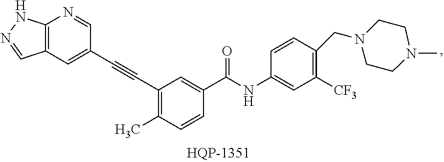 [SMILES: Cc1ccc(C(=O)Nc2ccc(CN3CCN(C)CC3)c(C(F)(F)F)c2)cc1C#Cc1cnc2[nH]ncc2c1]
wherein the allosteric inhibitor is asciminib.

3.-4. (canceled)

5. The method of claim 1, wherein the hematological malignancy is leukemia.

6. The method of claim 1, wherein the hematological malignancy is chronic myelogenous leukemia.

7. The method of claim 6, wherein the method is in the treatment of the patient with chronic myeloid leukemia resistant to current tyrosine kinase inhibitor therapies.

8. The method of claim 7, wherein the patient with chronic myeloid leukemia resistant to the current tyrosine kinase inhibitor therapies is caused by BCR-ABL mutations.

9. The method of claim 8, where the BCR-ABL mutation is T3151, E255K/V, G250E, H396P, M351T, Q252H, Y253F/H, or BCR-ABL^{WT} mutations.

10. The method of claim 8, where the BCR-ABL mutation is T3151 mutation.

11.-13. (canceled)

14. A method of inhibiting BCR-ABL mutants comprising contacting BCR-ABL mutants with a) HQP-1351 or a pharmaceutically acceptable salt thereof; and b) asciminib;
wherein HQP-1351 has the following structure:

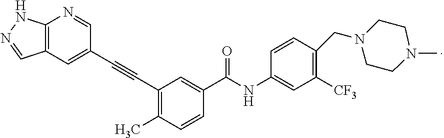 [SMILES: Cc1ccc(C(=O)Nc2ccc(CN3CCN(C)CC3)c(C(F)(F)F)c2)cc1C#Cc1cnc2[nH]ncc2c1]

15.-16. (canceled)

17. The method of claim 14, wherein the contact is in vitro or in vivo.

18. The method of claim 14, wherein the contact is in a patient with chronic myeloid leukemia resistant to current tyrosine kinase inhibitor therapies.

19. The method of claim 18, wherein the patient with chronic myeloid leukemia resistant to the current tyrosine kinase inhibitor therapies is caused by BCR-ABL mutations.

20. The method of claim 19, wherein the BCR-ABL mutation is T3151, E255K/V, G250E, H396P, M351T, Q252H, Y253F/H, or BCR-ABL^{WT} mutations.

21. The method of claim 19, wherein the BCR-ABL mutation is T3151.

22. A method of treating cancer comprising co-administering to a subject in need thereof:
a) HQP-1351 or a pharmaceutically acceptable salt thereof; and
b) an immune checkpoint molecule selected from PD-1, PD-L1, PD-L2, CTLA-4, TIM-3, LAG3, CD160, 2B4, TGFβ, VISTA, BTLA, TIGIT and LAIR1;
wherein the cancer is breast cancer, cervical cancer, ovarian cancer, endometrial cancer, prostate cancer, colon cancer, bladder cancer, bone metastasis, colorectal cancer, esophagus cancer, head and neck cancer, small cell lung cancer, non-small cell lung carcinoid tumor, or stomach carcinoma, wherein HQP-1351 has the following structure:

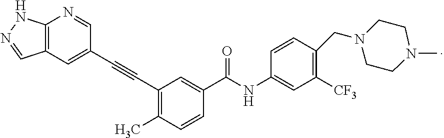 [SMILES: Cc1ccc(C(=O)Nc2ccc(CN3CCN(C)CC3)c(C(F)(F)F)c2)cc1C#Cc1cnc2[nH]ncc2c1]

23.-25. (canceled)

26. The method of claim 22, wherein the immune checkpoint molecule is pembrolizumab, ipilimumab, nivolumab, atezolizumab, avelumab, durvalumab, cemiplimab, lirilumab, tremelimumab, or pidilizumab.

27. The method of claim 22, wherein the immune checkpoint molecule is AMP-224, AMP-514, BGB-A317, cemiplimab, JS001, PDR-001, PF-06801591, IBI-308, pidilizumab, SHR-1210, or TSR-042.

28-37. (canceled)

38. A method of inhibiting proliferation of Ph+ ALL SUP-B15 cells or inducing apoptosis of primary pre-B ALL cells and ALL cells comprising administering to a subject in need thereof HQP-1351 or a pharmaceutically acceptable salt thereof, wherein HQP-1351 has the following structure:

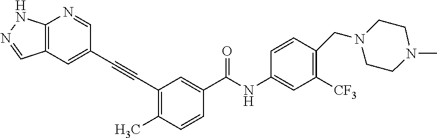 [SMILES: Cc1ccc(C(=O)Nc2ccc(CN3CCN(C)CC3)c(C(F)(F)F)c2)cc1C#Cc1cnc2[nH]ncc2c1]

39. (canceled)

40. A method of treating renal cancer comprising co-administering to a subject in need thereof:
a) HQP-1351 or a pharmaceutically acceptable salt thereof; and
b) an anti-PD-1 antibody selected from InVivoMab anti-mouse PD-1(CD279), wherein HQP-1351 has the following structure:

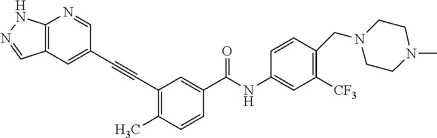 [SMILES: Cc1ccc(C(=O)Nc2ccc(CN3CCN(C)CC3)c(C(F)(F)F)c2)cc1C#Cc1cnc2[nH]ncc2c1]

41.-42. (canceled)

43. The method of claim 40, wherein the renal cancer is renal cell carcinoma, clear cell renal cell carcinoma (ccRCC), papillary renal cell carcinoma (PRCC), clear cell papillary renal cell carcinoma, or chromophobe renal cell carcinoma.

44.-47. (canceled)


SEQUENCE LISTING (6 sequences)

SEQ ID NO: 1 - DNA, 20 bp, Homo sapiens
  source 1..20: mol_type = genomic DNA
gagcggctccagattgtcaa

SEQ ID NO: 2 - DNA, 21 bp, Homo sapiens
  source 1..21: mol_type = genomic DNA
ctggggatgtagcctgtctgt

SEQ ID NO: 3 - DNA, 20 bp, Homo sapiens
  source 1..20: mol_type = genomic DNA
tatggtggtgccgactacaa

SEQ ID NO: 4 - DNA, 20 bp, Homo sapiens
  source 1..20: mol_type = genomic DNA
tgcttgtccagatgacttcg

SEQ ID NO: 5 - DNA, 21 bp, Homo sapiens
  source 1..21: mol_type = genomic DNA
ggtgaaggtcggagtcaacgg

SEQ ID NO: 6 - DNA, 22 bp, Homo sapiens
  source 1..22: mol_type = genomic DNA
cctggaagatggtgatgggatt